(19) United States
(12) Patent Application Publication
(10) Pub. No.: US 2026/0347280 A1
(43) Pub. Date: Nov. 5, 2026

(54) DEUTERATED HINOKITIOL DERIVATIVES

(71) Applicant: The Board of Trustees of the University of Illinois, Urbana, IL (US)

(72) Inventors: Martin D. Burke, Champaign, IL (US); Andrew D. Blake, Rockville, MD (US); Christopher Kolste Rakowski, Urbana, IL (US)

(21) Appl. No.: 19/116,047

(22) PCT Filed: Oct. 2, 2023
(86) PCT No.: PCT/US2023/034258

§ 371 Date: Mar. 27, 2025

Related U.S. Application Data
(60) Provisional application No. 63/411,738, filed on Sep. 30, 2022.

(51) Int. Cl. C07B 59/00; A61K 9/1277; A61K 31/122; A61K 31/341; A61K 31/351; A61K 31/4412; A61K 45/06; C07C 49/717; C07C 49/753; C07D 307/12; C07D 309/06

(52) U.S. Cl. CPC C07B 59/001; A61K 9/1277; A61K 31/122; A61K 31/341; A61K 31/351; A61K 31/4412; A61K 45/06; C07B 59/002; C07C 49/717; C07C 49/753; C07D 307/12; C07D 309/06; C07B 2200/05; C07C 2601/02; C07C 2601/04; C07C 2601/18

(57) ABSTRACT

Disclosed are deuterated derivatives of tropolone useful for the treatment of diseases or disorders, e.g., involving deficiencies in iron transport.

### RELATED APPLICATIONS

This application claims the benefit of priority to U.S. Provisional Application No. 63/411,738, filed Sep. 30, 2022.

### STATEMENT OF GOVERNMENT SUPPORT

This invention was made with government support under 1R01HL140526 awarded by the National Institutes of Health. The government has certain rights in the invention.

### BACKGROUND

Hinokitiol, a small molecule natural product, has been shown to promote transmembrane iron mobilization by reversibly forming a 3:1 complex with iron. Hinokitiol restores iron homeostasis in a range of different model systems, including divalent metal transporter 1 (DMT1−), mitoferrin 1 (MFRN1−), and ferroportin 1 (FPN1−) deficient cells and animals. Hinokitiol, which is it itself not inherently site- or direction-selective, achieves site- and direction-selective mobilization of iron by leveraging the electrochemical gradient that forms in the absence of iron transport protein function.

There is, however, a key limitation of hinokitiol, which diminishes its potential for clinical translation. In differentiated shDMT1 monolayers, it has been discovered that hinokitiol effectively mobilizes iron at low-to-intermediate concentrations, but at higher concentrations, a substantial decrease in transmembrane mobilizing activity was observed. A series of studies was conducted to identify derivatives of hinokitiol, such as those disclosed herein, which mobilize iron over a much wider range of concentrations in vitro. These compounds also mobilize iron and can treat anemia of inflammation in vivo.

En route to these improved derivatives, the loss of iron mobilization at higher concentrations of hinokitiol to the formation of higher-order aggregates in aqueous media was identified. These aggregates then restrict the amount of free tropolone:iron complexes that are capable of transmembrane mobilization of iron. To understand how these complexes might interact with one another in these aggregates, several crystal structures of hinokitiol:M (M=Fe, Al, Cu) complexes were obtained. In these crystal structures, the isopropyl groups of these complexes make key van der Waals interactions that might drive the formation of aggregates in aqueous media. Using a constitutional isomer of hinokitiol which was less prone to aggregation, the van der Waals interactions made by the isopropyl group differed significantly.

Deuterium is an isotope of hydrogen which includes a neutron in the nucleus, doubling the atomic weight. This lowers the zero-point energy and decreases the van der Waals radii of deuterated compounds relative to their hydrogenated counterparts. However, literature concerning the effect of deuteration of pharmaceuticals mostly focuses on harnessing the kinetic isotope effect to improve the pharmacokinetic profile of a drug. Some studies on deuterated materials have reported alterations in boiling point, dimerization behavior, among others. Few, if any, systematic studies exist which explain the relationship between deuteration and altered biophysical behavior of a pharmaceutical agent. Thus, it was unclear whether deuteration would have any impact on this aggregation phenomena, and it was further uncertain whether any impact would be positive or negative. Using substituted tropolones, it was discovered that deuteration of the side chains of tropolonoids can increase both potency and tolerability, thereby improving their potential to act as molecular prosthetics for metal ion transporters.

### SUMMARY OF THE INVENTION

In certain aspects, provided herein are compounds of Formula I:

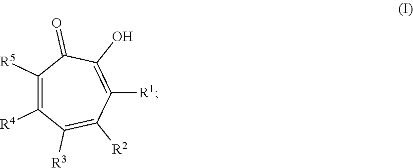

or pharmaceutically acceptable salts thereof;
wherein:
R¹ is selected from the group consisting of H, C₁-C₅ alkyl, cycloalkyl, heterocycloalkyl, —O—C₁-C₅ alkyl, —O-cycloalkyl, and —O-heterocycloalkyl;
R² is selected from the group consisting of H, C₁-C₅ alkyl, cycloalkyl, heterocycloalkyl, —O—C₁-C₅ alkyl, —O-cycloalkyl, and —O-heterocycloalkyl; and
R³ is selected from the group consisting of H, C₁-C₅ alkyl, cycloalkyl, heterocycloalkyl, —O—C₁-C₅ alkyl, —O-cycloalkyl, and —O-heterocycloalkyl;
R⁴ is selected from the group consisting of H, C₁-C₅ alkyl, cycloalkyl, heterocycloalkyl, —O—C₁-C₅ alkyl, —O-cycloalkyl, and —O-heterocycloalkyl; and
R⁵ is selected from the group consisting of H, halo, C₁-C₅ alkyl, cycloalkyl, heterocycloalkyl, —O—C₁-C₅ alkyl, —O-cycloalkyl, and —O-heterocycloalkyl;
wherein when R¹, R², R³, R⁴, or R⁵ is C₁-C₅ alkyl, cycloalkyl, heterocycloalkyl, —O—C₁-C₅ alkyl, —O-cycloalkyl, or —O-heterocycloalkyl, at least one hydrogen atom in said C₁-C₅ alkyl, cycloalkyl, heterocycloalkyl, —O—C₁-C₅ alkyl, —O-cycloalkyl, or —O-heterocycloalkyl is replaced with a deuterium atom; and
wherein at least one of R¹, R², R³, and R⁴ is not H.

In further aspects, provided herein are pharmaceutical compositions, comprising a compound of the disclosure, or a pharmaceutically acceptable salt thereof; and a pharmaceutically acceptable carrier or excipient.

In yet further aspects, provided herein are methods of treating a disease or condition characterized by decreased ferroportin, comprising administering to a subject in need thereof a therapeutically effective amount of a compound or pharmaceutical composition of the disclosure.

In still further aspects, provided herein are methods of treating a disease or condition characterized by decreased iron transport, comprising administering to a subject in need thereof a therapeutically effective amount of a compound or pharmaceutical composition of the disclosure.

In certain aspects, provided herein are methods of treating a disease or condition characterized by a defect or deficiency in an iron transporter, comprising administering to a subject in need thereof a therapeutically effective amount of a compound or pharmaceutical composition of the disclosure.

In further aspects, provided herein are methods of treating a disease or condition characterized by iron accumulation, comprising administering to a subject in need thereof a therapeutically effective amount of a compound or pharmaceutical composition of the disclosure.

In yet further aspects, provided herein are methods of treating a disease or condition characterized by iron misdistribution, comprising administering to a subject in need thereof a therapeutically effective amount of a compound or pharmaceutical composition of the disclosure.

In certain aspects, provided herein are methods of treating a neurodegenerative disorder, comprising administering to a subject in need thereof a therapeutically effective amount of a compound or pharmaceutical composition of the disclosure.

### BRIEF DESCRIPTION OF THE DRAWINGS

FIG. 1 depicts a general synthetic scheme for accessing deuterated tropolone derivatives.

FIG. 2A depicts data for an evaluation of methyl-d₀ and methyl-d₃ tropolones. Iron mobilized by deuterated derivatives increases at low concentrations, and remains high at extreme concentrations.

FIG. 2B depicts data for an evaluation of methyl-d₀ and methyl-d₃ tropolones. Deuterated derivatives are significantly better tolerated in H9C2 cells.

FIG. 2C depicts data for an evaluation of methyl-d₀ and methyl-d₃ tropolones. Deuterated derivatives are better tolerated in K562 cells.

FIG. 3A depicts results of treatment of fpn-1.2KO C. elegans with various compounds for neurodegeneration scoring and iron level measurement: Treatment scheme of fpn-1.2KO; Pdat::GFP worms treated with various concentrations of DFP, Hino, and AMB-1269. Dopaminergic neurodegeneration was scored blinded by phenotypic analyses (below).

FIG. 3B depicts dopaminergic neurodegeneration scoring of fpn-1.2KO worms treated with DFP.

FIG. 3C depicts dopaminergic neurodegeneration scoring of fpn-1.2KO worms treated with Hino.

FIG. 3D depicts dopaminergic neurodegeneration scoring of fpn-1.2KO worms treated with FeM-1269.

FIG. 3E depicts a Treatment scheme of fpn-1.2KO; Pftn-1::GFP worms treated with various concentrations of DFP, Hino, and FeM-1269. ASI neurons (below) which expresses GFP-tagged ferritin levels.

FIG. 3F depicts Fluorescence levels of fpn-1.2KO worms treated with DFP. * P<0.05, ** P<0.01, *** P<0.001, and **** P<0.0001 by one-way ANOVA.

FIG. 3G depicts Fluorescence levels of fpn-1.2KO worms treated with Hino. * P<0.05, ** P<0.01, *** P<0.001, and **** P<0.0001 by one-way ANOVA.

FIG. 3H depicts Fluorescence levels of fpn-1.2KO worms treated with FeM-1269. * P<0.05, ** P<0.01, *** P<0.001, and **** P<0.0001 by one-way ANOVA.

FIG. 4A depicts data from flatiron mice experiments: Flatiron mice display increased anxiety and reduced exploratory activity. WT and flatiron mice were subjected to elevated plus maze task for assessment of anxiety-like behavior, including total distance traveled in whole maze and average velocity.

FIG. 4B depicts data from flatiron mice experiments: Flatiron mice display increased anxiety and reduced exploratory activity. WT and flatiron mice were subjected to elevated plus maze task for assessment of anxiety-like behavior, including time spent in open arms and center zone.

FIG. 4C depicts data from flatiron mice experiments: Flatiron mice display increased anxiety and reduced exploratory activity. WT and flatiron mice were subjected to elevated plus maze task for assessment of anxiety-like behavior, including rearing frequency and duration. * P<0.05 by Student's t-test.

FIG. 4D depicts ICP-MS measurement of brain iron level in flatiron mice after acute treatment of Hino through intraperitoneal (IP) injection. ** P<0.01, *** P<0.001 by one-way ANOVA.

FIG. 4E depicts ICP-MS measurement of brain iron level in flatiron mice after chronic treatment of Hino through intraperitoneal (IP) injection. ** P<0.01, *** P<0.001 by one-way ANOVA.

FIG. 5 depicts crystallographic data showing hinokitiol forms a 3:1 complex with Fe(III) for transmembrane mobilization. Hinokitiol in crystal packing structure holds isopropyl sidechains from neighboring complexes in proximity.

FIG. 6 depicts a schematic showing deuteration is hypothesized to reduce intercomplex van der Waals interactions, restoring transport ability at higher concentrations for pro-aggregative derivatives.

FIG. 7A depicts cell-free iron efflux data showing per-deuteration of hinokitiol promotes higher concentration transport similar to that observed with 3,5,7-tri-methyl tropolone.

FIG. 7B depicts dynamic light scattering data showing per-deuteration of hinokitiol reduces aggregation.

FIG. 8A depicts crystallographic data for hinokitiol showing isopropyl-isopropyl interaction.

FIG. 8B depicts cell-free iron efflux data showing per-deuteration of isopropyl isomer GT does not influence transport at higher concentrations, consistent with limited specific isopropyl-isopropyl interaction seen in crystal structure.

FIG. 9A depicts dynamic light scattering data (deflected count) showing per-deuteration of methyl sidechains does not significantly affect aggregation due to lack of discrete interaction and limited aggregation of the protio parent compound.

FIG. 9B depicts dynamic light scattering data (average size) showing per-deuteration of methyl sidechains does not significantly affect aggregation due to lack of discrete interaction and limited aggregation of the protio parent compound.

FIG. 10A depicts cell-free iron efflux data showing similar iron mobilization capacity between 3-methyltropolone and 3-methyl-(d₃)-tropolone.

FIG. 10B depicts cell-free iron efflux data showing similar iron mobilization capacity between 4-methyltropolone and 4-methyl-(d₃)-tropolone.

FIG. 10C depicts cell-free iron efflux data showing similar iron mobilization capacity between 5-methyltropolone and 5-methyl-(d₃)-tropolone.

FIG. 10D depicts cell-free iron efflux data showing similar iron mobilization capacity between 3,5-dimethyltropolone and 3,5-dimethyl-(d₆)-tropolone.

FIG. 11 depicts cell-free iron efflux data showing per-deuteration extends transport window of pro-aggregative 4-n-buytl-(d₉)-tropolone compared to 4-n-buytltropolone.

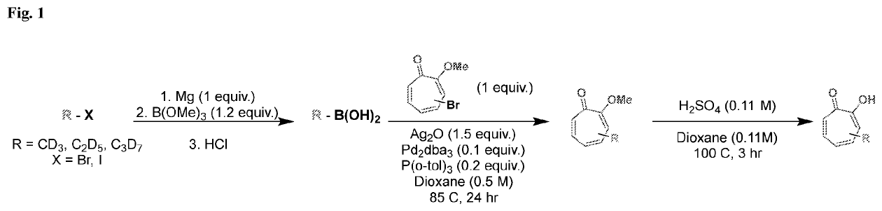

### DETAILED DESCRIPTION OF THE INVENTION

Iron is biologically essential, but also potentially toxic; as such it is tightly controlled at cell and systemic levels to prevent both deficiency and overload. Iron overload can manifest in two primary ways: (1) an excess of iron in circulation, and (2) an accumulation of iron in tissues. The latter may also be referred to as iron misdistribution or maldistribution. Iron chelators treat iron overload and enhance iron excretion by binding to iron in the blood, decreasing the amount of iron in circulation. Iron mobilizers pull iron out of tissues, and can therefore redistribute iron from sites of accumulation to sites of deficiency. Such iron mobilizers can collaborate with iron-binding proteins to restore normal iron physiology. Current treatments primarily address excess iron in the blood.

Iron transport throughout living systems is achieved via the concerted activity of passive and active transport proteins. Disruption of iron homeostasis can arise from diseases induced by dysfunctional activity in just one of these proteins. Hinokitiol (β-thujaplicin), a natural product, has been shown to autonomously replace ion transport protein function in disease models via forming lipophilic 3:1 complex with iron and leveraging transmembrane concentration gradients to redistribute iron. However, hinokitiol is predisposed at higher concentrations to form higher-order aggregates, limiting the efficacious mobilization range. From x-ray crystal structure of the complex, the β-substituted isopropyl group of adjacent complexes is proposed to promote aggregation through proximity-driven attractive van der Waals interactions. Reduction of these interactions whilst maintaining adequate lipophilicity for transmembrane mobilization will generally increase mobilization efficacy range, as observed in synthetic design principles for new tropolone derivatives. Notably, these new derivatives are usually structurally distinct from aggregative compounds, with no alternative modification means to transition an aggregative compound into one that has a lessened tendency to aggregate but holds the same structural design.

Deuteration of small molecules is usually used to alter pharmacokinetics due to the Kinetic Isotope Effect (KIE). The literature is sparse and mixed when it comes to the effects of deuteration on intermolecular van der Waals-type interactions. Moreover, whereas normal primary KIE can result in a rate ratio value of 1 to 8, non-covalent deuterium isotope effects are reported to be very modest, in the range of 1-1.5. It was thus unclear if deuteration could help address the aggregation-based loss of activity observed with tropolones such as hinokitiol, which was suggested by preliminary evidence to be driven by higher-order application. However, sparse applications in supramolecular host-guest interactions, rotational barriers, chromatography, and self-association were intriguing, and suggested to us that albeit slight, impactful physical chemical differences on aggregation might be realizable by deuteration, especially in the case of perdeuteration where these individual effects might be amplified. Thus, despite strong literature precedent, we decided to test experimentally whether deuteration of aggregation-prone small molecule metal mobilizers could improve availability of the active transport complex at higher concentrations through complex association interruption. We specifically tested whether per-deuteration of pro-aggregative sidechains might largely maintain their lipophilicity while increasing in vitro activity range compared to parent compounds due to a potential effect from deuteration interrupting intercomplex van der Waals interactions, thus reducing small molecule higher-order aggregation with minimal structural perturbation.

To first test the application of deuteration to iron mobilizers, hinokitiol-d₇ was synthesized utilizing a Negishi coupling of 2-iodopropane-d₇ to a modular bromotropolone scaffold. Hinokitiol was utilized as a highly aggregative standard, with hinokitiol-d₇ the comparison isotopomer containing a perdeuterated isopropyl sidechain. In Fe3+ liposomal efflux assays, hinokitiol-d₇ had significantly greater iron transport capability at higher concentrations (50-150 μM) when compared to hinokitiol, which begins to aggregate. This transportation capacity is similar to the non-isopropyl constitutional isomer control Tri-Methyl which aggregates less yet has the same cLogP as hinokitiol, suggesting that hinokitiol-d₇ is also aggregating to a lesser degree. This high concentration transport ability level is not comparable with most other derivatives, an interesting development for developing improved iron mobilizers. Lesser aggregation is supported by dynamic light scattering studies, which show that hinokitiol-d₇ from 20-100 μM with equimolar iron aggregates less via lesser photon deflection counts and particle size. Interestingly, an isopropyl constitutional isomer, GT (γ-thujaplicin), which has previously been shown to have less aggregation (and a different crystal packing limiting intercomplex isopropyl proximity) than hinokitiol demonstrates no deuteration effect on cell-free iron transport. This suggests that deuteration of non-interacting sidechains holds no effect on transmembrane iron transport. This is supported by the non-interacting methyl and methyl-d₃ variants at all ring positions, which show no actionable differences in cell-free iron transport and all have low aggregative tendencies. These methyl variants also show no significant differences in shDMT1-Caco-2 monolayer transport experiments that were able to be ran, signifying an extension of the cell-free trends to a biotic system. Multiple substituted derivatives such as 3′5DiMe also do not show any effect upon deuteration, even as the number of deuterium substitutions increases, since the protio parent compound is non-aggregating in nature.

To support this differential deuteration effect dependent on parent compound aggregation tendencies, a highly aggregating compound 4 nBu and its per-deuterated isotopomer pair were synthesized and tested in the liposome efflux assay. 4 nBu has a shortened effective transmembrane mobilization concentration range due to higher order aggregation, ceasing noticeable transport at 40 μM of compound. 4 nBu-d9, similarly to Hinokitiol-d7, maintains transport levels at concentrations when the parent compound suffers from limiting higher order aggregation. Strikingly, in this case this range is extended to 75 μM of compound that demonstrates noticeable transmembrane transport of iron before dropping to baseline, a sign that deuteration can be strategically utilized as a new design principle to extend the range of effective concentrations with aggregating tropolone derivatives. In certain embodiments, further primary alkyl derivatives may be synthesized utilizing commercial per-deuterated halides that are transformed to the corresponding boronic acid through Grignard-promoted addition of trimethyl borane and then utilized in Suzuki-Miyaura cross coupling to the bromotropolone scaffolds.

The present disclosure is based on the surprising discovery that deuteration of the side chains of tropolonoids can increase both potency and tolerability, thereby improving their potential to act as molecular prosthetics for metal ion transporters. The compounds of the disclosure are useful for treating diseases caused by iron misdistribution.

### DEFINITIONS

The term “organic moiety” as used herein refers to a singly-valent group containing one or more carbon atoms. The organic moiety may be aromatic or may be derived from a hydrocarbon. The organic moiety may comprise one or more heteroatoms, one or more units of unsaturation, and/or one or more functional groups. The organic moiety may be substituted.

The term “alkyl” as used herein is a term of art and refers to saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight-chain or branched-chain alkyl has about 30 or fewer carbon atoms in its backbone (e.g., C₁-C₃₀ for straight chain, C₃-C₃₀ for branched chain), and alternatively, about 20 or fewer, or 10 or fewer. In certain embodiments, the term “alkyl” refers to a C₁-C₁₀ alkyl group. In certain embodiments, the term “alkyl” refers to a C₁-C₆ alkyl group, for example a C₁-C₆ straight-chain alkyl group. In certain embodiments, the term “alkyl” refers to a C₃-C₁₂ branched-chain alkyl group. In certain embodiments, the term “alkyl” refers to a C₃-C₈ branched-chain alkyl group. Representative examples of alkyl include, but are not limited to, methyl, ethyl, n-propyl, iso-propyl, n-butyl, sec-butyl, iso-butyl, tert-butyl, n-pentyl, isopentyl, neopentyl, and n-hexyl.

The term “cycloalkyl” means mono- or bicyclic or bridged saturated carbocyclic rings, each having from 3 to 12 carbon atoms. Certain cycloalkyls have from 5-12 carbon atoms in their ring structure, and may have 6-10 carbons in the ring structure. Preferably, cycloalkyl is (C₃-C₇)cycloalkyl, which represents a monocyclic saturated carbocyclic ring, having from 3 to 7 carbon atoms. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems include bridged monocyclic rings and fused bicyclic rings. Bridged monocyclic rings contain a monocyclic cycloalkyl ring where two non-adjacent carbon atoms of the monocyclic ring are linked by an alkylene bridge of between one and three additional carbon atoms (i.e., a bridging group of the form —(CH₂)_{w}—, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. The bridged or fused bicyclic cycloalkyl is attached to the parent molecular moiety through any carbon atom contained within the monocyclic cycloalkyl ring. Cycloalkyl groups are optionally substituted. In certain embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted.

The term “(cycloalkyl)alkyl” as used herein refers to an alkyl group substituted with one or more cycloalkyl groups. An example of (cycloalkyl)alkyl is cyclohexylmethyl group.

The term “heterocycloalkyl” as used herein refers to a radical of a non-aromatic ring system, including, but not limited to, monocyclic, bicyclic, and tricyclic rings, which can be completely saturated or which can contain one or more units of unsaturation, for the avoidance of doubt, the degree of unsaturation does not result in an aromatic ring system, and having 3 to 12 atoms including at least one heteroatom, such as nitrogen, oxygen, or sulfur. For purposes of exemplification, which should not be construed as limiting the scope of this invention, the following are examples of heterocyclic rings: aziridinyl, azirinyl, oxiranyl, thiiranyl, thiirenyl, dioxiranyl, diazirinyl, diazepanyl, 1,3-dioxanyl, 1,3-dioxolanyl, 1,3-dithiolanyl, 1,3-dithianyl, imidazolidinyl, isothiazolinyl, isothiazolidinyl, isoxazolinyl, isoxazolidinyl, azetyl, oxetanyl, oxetyl, thietanyl, thietyl, diazetidinyl, dioxetanyl, dioxetenyl, dithietanyl, dithietyl, dioxalanyl, oxazolyl, thiazolyl, triazinyl, isothiazolyl, isoxazolyl, azepines, azetidinyl, morpholinyl, oxadiazolinyl, oxadiazolidinyl, oxazolinyl, oxazolidinyl, oxopiperidinyl, oxopyrrolidinyl, piperazinyl, piperidinyl, pyranyl, pyrazolinyl, pyrazolidinyl, pyrrolinyl, pyrrolidinyl, quinuclidinyl, thiomorpholinyl, tetrahydropyranyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. A heterocycloalkyl group is optionally substituted by one or more substituents as described below.

The term “(heterocycloalkyl)alkyl” as used herein refers to an alkyl group substituted with one or more heterocycloalkyl (i.e., heterocyclyl) groups.

The term “alkenyl” as used herein means a straight or branched chain hydrocarbon radical containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl. The unsaturated bond(s) of the alkenyl group can be located anywhere in the moiety and can have either the (Z) or the (E) configuration about the double bond(s).

The term “alkynyl” as used herein means a straight or branched chain hydrocarbon radical containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “alkylene” is art-recognized, and as used herein pertains to a diradical obtained by removing two hydrogen atoms of an alkyl group, as defined above. In one embodiment an alkylene refers to a disubstituted alkane, i.e., an alkane substituted at two positions with substituents such as halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like. That is, in one embodiment, a “substituted alkyl” is an “alkylene”.

The term “amino” is a term of art and as used herein refers to both unsubstituted and substituted amines, e.g., a moiety that may be represented by the general formulas:

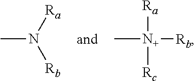

wherein Rₐ, R_{b}, and R_{c} each independently represent a hydrogen, an alkyl, an alkenyl, —(CH₂)ₓ—Rₐ, or Rₐ and R_{b}, taken together with the N atom to which they are attached complete a heterocycle having from 4 to 8 atoms in the ring structure; R_{d} represents an aryl, a cycloalkyl, a cycloalkenyl, a heterocyclyl or a polycyclyl; and x is zero or an integer in the range of 1 to 8. In certain embodiments, only one of Rₐ or R_{b} may be a carbonyl, e.g., Rₐ, R_{b}, and the nitrogen together do not form an imide. In other embodiments, Rₐ and R_{b} (and optionally R_{c}) each independently represent a hydrogen, an alkyl, an alkenyl, or —(CH₂)ₓ—R_{d}. In certain embodiments, the term “amino” refers to —NH₂.

In certain embodiments, the term “alkylamino” refers to —NH(alkyl).

In certain embodiments, the term “dialkylamino” refers to —N(alkyl)₂.

The term “amido”, as used herein, means-NHC(═O)—, wherein the amido group is bound to the parent molecular moiety through the nitrogen. Examples of amido include alkylamido such as CH₃C(═O)N(H)— and CH₃CH₂C(═O)N(H)—.

The term “acyl” is a term of art and as used herein refers to any group or radical of the form RCO— where R is any organic group, e.g., alkyl, aryl, heteroaryl, aralkyl, and heteroaralkyl. Representative acyl groups include acetyl, benzoyl, and malonyl.

The term “aminoalkyl” as used herein refers to an alkyl group substituted with one or more one amino groups. In one embodiment, the term “aminoalkyl” refers to an aminomethyl group.

The term “aminoacyl” is a term of art and as used herein refers to an acyl group substituted with one or more amino groups.

The term “aminothionyl” as used herein refers to an analog of an aminoacyl in which the O of RC(O)— has been replaced by sulfur, hence is of the form RC(S)—.

The term “phosphoryl” is a term of art and as used herein may in general be represented by the formula:

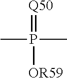

wherein Q50 represents S or O, and R59 represents hydrogen, a lower alkyl or an aryl; for example, —P(O)(OMe)- or —P(O)(OH)₂. When used to substitute, e.g., an alkyl, the phosphoryl group of the phosphorylalkyl may be represented by the general formulas:

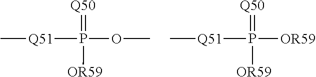

wherein Q50 and R59, each independently, are defined above, and Q51 represents O, S or N; for example, —O—P(O)(OH)OMe or —NH—P(O)(OH)₂. When Q50 is S, the phosphoryl moiety is a “phosphorothioate.”

The term “aminophosphoryl” as used herein refers to a phosphoryl group substituted with at least one amino group, as defined herein; for example, —P(O)(OH)NMe₂.

The term “azide” or “azido”, as used herein, means an —N₃ group.

The term “carbonyl” as used herein refers to —C(═O)—.

The term “thiocarbonyl” as used herein refers to —C(═S)—.

The term “alkylphosphoryl” as used herein refers to a phosphoryl group substituted with at least one alkyl group, as defined herein; for example, —P(O)(OH)Me.

The term “alkylthio” as used herein refers to alkyl-S—. The term “(alkylthio)alkyl” refers to an alkyl group substituted by an alkylthio group.

The term “carboxy”, as used herein, means a —CO₂H group.

The term “aryl” is a term of art and as used herein refers to includes monocyclic, bicyclic and polycyclic aromatic hydrocarbon groups, for example, benzene, naphthalene, anthracene, and pyrene. Typically, an aryl group contains from 6-10 carbon ring atoms (i.e., (C₆-C₁₀) aryl). The aromatic ring may be substituted at one or more ring positions with one or more substituents, such as halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like. The term “aryl” also includes polycyclic ring systems having two or more cyclic rings in which two or more carbons are common to two adjoining rings (the rings are “fused rings”) wherein at least one of the rings is an aromatic hydrocarbon, e.g., the other cyclic rings may be cycloalkyls, cycloalkenyls, cycloalkynyls, aryls, heteroaryls, and/or heterocyclyls. In certain embodiments, the term “aryl” refers to a phenyl group.

The term “arylene” means a diradical obtained by removing two hydrogen atoms of an aryl group, as defined above. In certain embodiments an arylene refers to a disubstituted arene, i.e., an arene substituted at two positions with substituents such as halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluoromethyl), cyano, or the like. That is, in certain embodiments, a “substituted aryl” is an “arylene”.

The term “heteroaryl” is a term of art and as used herein refers to a monocyclic, bicyclic, and polycyclic aromatic group having 3 to 12 total atoms including one or more heteroatoms such as nitrogen, oxygen, or sulfur in the ring structure. Exemplary heteroaryl groups include azaindolyl, benzo(b)thienyl, benzimidazolyl, benzofuranyl, benzoxazolyl, benzothiazolyl, benzothiadiazolyl, benzotriazolyl, benzoxadiazolyl, furanyl, imidazolyl, imidazopyridinyl, indolyl, indolinyl, indazolyl, isoindolinyl, isoxazolyl, isothiazolyl, isoquinolinyl, oxadiazolyl, oxazolyl, purinyl, pyranyl, pyrazinyl, pyrazolyl, pyridinyl, pyrimidinyl, pyrrolyl, pyrrolo[2,3-d]pyrimidinyl, pyrazolo[3,4-d]pyrimidinyl, quinolinyl, quinazolinyl, triazolyl, thiazolyl, thiophenyl, tetrahydroindolyl, tetrazolyl, thiadiazolyl, thienyl, thiomorpholinyl, triazolyl or tropanyl, and the like. The “heteroaryl” may be substituted at one or more ring positions with one or more substituents such as halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like. The term “heteroaryl” also includes polycyclic ring systems having two or more cyclic rings in which two or more carbons are common to two adjoining rings (the rings are “fused rings”) wherein at least one of the rings is an aromatic group having one or more heteroatoms in the ring structure, e.g., the other cyclic rings may be cycloalkyls, cycloalkenyls, cycloalkynyls, aryls, heteroaryls, and/or heterocyclyls.

The term “heteroarylene” means a diradical obtained by removing two hydrogen atoms of a heteroaryl group, as defined above. In certain embodiments an heteroarylene refers to a disubstituted heteroarene, i.e., a heteroarene substituted at two positions with substituents such as halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluoromethyl), cyano, or the like. That is, in certain embodiments, a “substituted heteroaryl” is an “heteroarylene”.

The term “aralkyl” or “arylalkyl” is a term of art and as used herein refers to an alkyl group substituted with an aryl group, wherein the moiety is appended to the parent molecule through the alkyl group.

The term “heteroaralkyl” or “heteroarylalkyl” is a term of art and as used herein refers to an alkyl group substituted with a heteroaryl group, appended to the parent molecular moiety through the alkyl group.

The term “alkoxy” as used herein means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxyalkyl” refers to an alkyl group substituted by an alkoxy group.

The term “alkoxycarbonyl” means an alkoxy group, as defined herein, appended to the parent molecular moiety through a carbonyl group, represented by —C(═O)—, as defined herein. Representative examples of alkoxycarbonyl include, but are not limited to, methoxycarbonyl, ethoxycarbonyl, and tert-butoxycarbonyl.

The term “alkylcarbonyl”, as used herein, means an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “arylcarbonyl”, as used herein, means an aryl group, as defined herein, appended to the parent molecular moiety through a carbonyl group, as defined herein. Representative examples of arylcarbonyl include, but are not limited to, benzoyl and (2-pyridinyl) carbonyl.

The term “alkylcarbonyloxy” and “arylcarbonyloxy”, as used herein, means an alkylcarbonyl or arylcarbonyl group, as defined herein, appended to the parent molecular moiety through an oxygen atom. Representative examples of alkylcarbonyloxy include, but are not limited to, acetyloxy, ethylcarbonyloxy, and tert-butylcarbonyloxy. Representative examples of arylcarbonyloxy include, but are not limited to phenylcarbonyloxy.

The term “alkenoxy” or “alkenoxyl” means an alkenyl group, as defined herein, appended to the parent molecular moiety through an oxygen atom. Representative examples of alkenoxyl include, but are not limited to, 2-propen-1-oxyl (i.e., CH₂—CH—CH₂—O—) and vinyloxy (i.e., CH₂═CH—O—).

The term “aryloxy” as used herein means an aryl group, as defined herein, appended to the parent molecular moiety through an oxygen atom.

The term “heteroaryloxy” as used herein means a heteroaryl group, as defined herein, appended to the parent molecular moiety through an oxygen atom.

The term “carbocyclyl” as used herein means a monocyclic or multicyclic (e.g., bicyclic, tricyclic, etc.) hydrocarbon radical containing from 3 to 12 carbon atoms that is completely saturated or has one or more unsaturated bonds, and for the avoidance of doubt, the degree of unsaturation does not result in an aromatic ring system (e.g., phenyl). Examples of carbocyclyl groups include 1-cyclopropyl, 1-cyclobutyl, 2-cyclopentyl, 1-cyclopentenyl, 3-cyclohexyl, 1-cyclohexenyl and 2-cyclopentenylmethyl.

The term “cyano” is a term of art and as used herein refers to —CN.

The term “halo” is a term of art and as used herein refers to —F, —Cl, —Br, or —I.

The term “haloalkyl” as used herein refers to an alkyl group, as defined herein, wherein some or all of the hydrogens are replaced with halogen atoms.

The term “hydroxy” is a term of art and as used herein refers to —OH.

The term “hydroxyalkyl”, as used herein, means at least one hydroxy group, as defined herein, is appended to the parent molecular moiety through an alkyl group, as defined herein. Representative examples of hydroxyalkyl include, but are not limited to, hydroxymethyl, 2-hydroxyethyl, 3-hydroxypropyl, 2,3-dihydroxypentyl, and 2-ethyl-4-hydroxyheptyl.

The term “silyl”, as used herein, includes hydrocarbyl derivatives of the silyl (H₃Si—) group (i.e., (hydrocarbyl)₃Si—), wherein a hydrocarbyl groups are univalent groups formed by removing a hydrogen atom from a hydrocarbon, e.g., ethyl, phenyl. The hydrocarbyl groups can be combinations of differing groups which can be varied in order to provide a number of silyl groups, such as trimethylsilyl (TMS), tert-butyldiphenylsilyl (TBDPS), tert-butyldimethylsilyl (TBS/TBDMS), triisopropylsilyl (TIPS), and [2-(trimethylsilyl) ethoxy]methyl (SEM).

The term “silyloxy”, as used herein, means a silyl group, as defined herein, is appended to the parent molecule through an oxygen atom.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, fragmentation, decomposition, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

In certain embodiments, the optional substituents can include, for example, halogen, haloalkyl, hydroxyl, carbonyl (such as carboxyl (—COOH), alkoxycarbonyl, formyl, or acyl), thiocarbonyl (such as thioester, thioacetate, or thioformate), alkoxyl, alkenyloxy, alkynyloxy, phosphoryl, phosphate, phosphonate, phosphinate, amino (including alkyl- and dialkylamino), amido, amidine, imine, cyano, nitro, oxo (═O), azido, sulfhydryl, alkylthio, sulfate, sulfonate, sulfamoyl, sulfonamido, sulfonyl, silyl, silyloxy, heterocycloalkyl, cycloalkyl, alkyl, alkenyl, alkynyl, aryl, heteroaryl, arylalkyl, or heteroarylalkyl group.

For purposes of the invention, the chemical elements are identified in accordance with the Periodic Table of the Elements, CAS version, Handbook of Chemistry and Physics, 67th Ed., 1986-87, inside cover.

Other chemistry terms herein are used according to conventional usage in the art, as exemplified by The McGraw-Hill Dictionary of Chemical Terms (ed. Parker, S., 1985), McGraw-Hill, San Francisco, incorporated herein by reference). Unless otherwise defined, all technical and scientific terms used herein have the same meaning as commonly understood by one of ordinary skill in the art to which this invention pertains.

The term “pharmaceutically acceptable salt” as used herein includes salts derived from inorganic or organic acids including, for example, hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluoroacetic, trichloroacetic, naphthalene-2-sulfonic, and other acids. Pharmaceutically acceptable salt forms can include forms wherein the ratio of molecules comprising the salt is not 1:1. For example, the salt may comprise more than one inorganic or organic acid molecule per molecule of base, such as two hydrochloric acid molecules per molecule of compound of Formula I. As another example, the salt may comprise less than one inorganic or organic acid molecule per molecule of base, such as two molecules of compound of Formula I per molecule of tartaric acid.

The terms “carrier” and “pharmaceutically acceptable carrier” as used herein refer to a diluent, adjuvant, excipient, or vehicle with which a compound is administered or formulated for administration. Non-limiting examples of such pharmaceutically acceptable carriers include liquids, such as water, saline, and oils; and solids, such as gum acacia, gelatin, starch paste, talc, keratin, colloidal silica, urea, and the like. In addition, auxiliary, stabilizing, thickening, lubricating, flavoring, and coloring agents may be used. Other examples of suitable pharmaceutical carriers are described in Remington's Pharmaceutical Sciences by E. W. Martin, herein incorporated by reference in its entirety.

The term “treat” as used herein means prevent, halt or slow the progression of, or eliminate a disease or condition in a subject. In one embodiment “treat” means halt or slow the progression of, or eliminate a disease or condition in a subject. In one embodiment, “treat” means reduce at least one objective manifestation of a disease or condition in a subject.

The term “effective amount” as used herein refers to an amount that is sufficient to bring about a desired biological effect.

The term “therapeutically effective amount” as used herein refers to an amount that is sufficient to bring about a desired therapeutic effect.

The term “inhibit” as used herein means decrease by an objectively measurable amount or extent. In various embodiments, “inhibit” means decrease by at least 5, 10, 20, 30, 40, 50, 60, 70, 80, 90, or 95 percent compared to relevant control. In one embodiment, “inhibit” means decrease 100 percent, i.e., halt or eliminate.

The term “subject” as used herein refers to a mammal. In various embodiments, a subject is a mouse, rat, rabbit, cat, dog, pig, sheep, horse, cow, or non-human primate. In one embodiment, a subject is a human.

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Also unless otherwise stated, when a position is designated specifically as “D” or “deuterium”, the position is understood to have deuterium at an abundance that is at least 3340 times greater than the natural abundance of deuterium, which is 0.015% (i.e., at least 50.1% incorporation of deuterium).

The term “isotopic enrichment factor” as used herein means the ratio between the isotopic abundance and the natural abundance of a specified isotope.

In various embodiments, compounds of this invention have an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly (anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino) ethanol, ethanolamine, ethylenediamine, N-methylglucamine, hydrabamine, 1H-imidazole, lithium, L-lysine, magnesium, 4-(2-hydroxyethyl) morpholine, piperazine, potassium, 1-(2-hydroxyethyl) pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, l-ascorbic acid, l-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Compounds of the Disclosure

In certain aspects, provided herein are compounds of Formula I:

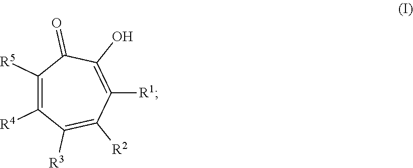

or pharmaceutically acceptable salts thereof;
wherein:
R¹ is selected from the group consisting of H, C₁-C₅ alkyl, cycloalkyl, heterocycloalkyl, —O—C₁-C₅ alkyl, —O-cycloalkyl, and —O-heterocycloalkyl;
R² is selected from the group consisting of H, C₁-C₅ alkyl, cycloalkyl, heterocycloalkyl, —O—C₁-C₅ alkyl, —O-cycloalkyl, and —O-heterocycloalkyl; and
R³ is selected from the group consisting of H, C₁-C₅ alkyl, cycloalkyl, heterocycloalkyl, —O—C₁-C₅ alkyl, —O-cycloalkyl, and —O-heterocycloalkyl;
R⁴ is selected from the group consisting of H, C₁-C₅ alkyl, cycloalkyl, heterocycloalkyl, —O—C₁-C₅ alkyl, —O-cycloalkyl, and —O-heterocycloalkyl; and
R⁵ is selected from the group consisting of H, halo, C₁-C₅ alkyl, cycloalkyl, heterocycloalkyl, —O—C₁-C₅ alkyl, —O-cycloalkyl, and —O-heterocycloalkyl;
wherein when R¹, R², R³, R₄, or R⁵ is C₁-C₅ alkyl, cycloalkyl, heterocycloalkyl, —O—C₁-C₅ alkyl, —O-cycloalkyl, or —O-heterocycloalkyl, at least one hydrogen atom in said C₁-C₅ alkyl, cycloalkyl, heterocycloalkyl, —O—C₁-C₅ alkyl, —O-cycloalkyl, or —O-heterocycloalkyl is replaced with a deuterium atom; and
wherein at least one of R¹, R², R³, and R⁴ is not H.

In certain embodiments, R¹ is selected from the group consisting of methyl, ethyl, propyl, and butyl. In further embodiments, R¹ is selected from the group consisting of CD₃, C₂D₅, C₃D₇, and C₄D₉. In yet further embodiments, R¹ is:

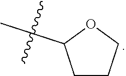 [SMILES: *C1CCCO1]

In still further embodiments, R¹ is:

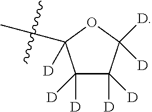

In certain embodiments, R¹ is:

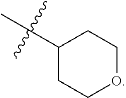 [SMILES: *C1CCOCC1]

In further embodiments, R¹ is:

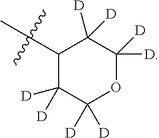

In yet further embodiments, R¹ is:

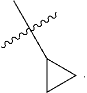 [SMILES: *C1CC1]

In still further embodiments, R¹ is:

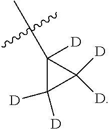

In certain embodiments, R¹ is selected from the group consisting of —OMe, —OEt, —OPr, and —OBu. In further embodiments, R¹ is selected from the group consisting of —OCD₃, —OC₂D₅, —OC₃D₇, and —OC₄D₉. In yet further embodiments, R¹ is:

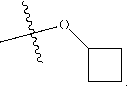 [SMILES: *OC1CCC1]

In still further embodiments, R¹ is:

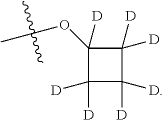

In certain embodiments, R² is selected from the group consisting of methyl, ethyl, propyl, and butyl. In further embodiments, R² is selected from the group consisting of CD₃, C₂D₅, C₃D₇, and C₄D₉. In yet further embodiments, R² is:

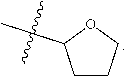 [SMILES: *C1CCCO1]

In still further embodiments, R² is:

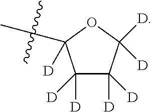

In certain embodiments, R² is:

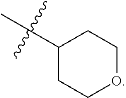 [SMILES: *C1CCOCC1]

In further embodiments, R² is:

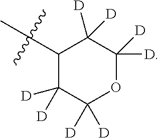

In yet further embodiments, R² is:

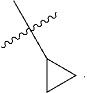 [SMILES: *C1CC1]

In still further embodiments, R² is:

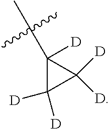

In certain embodiments, R² is selected from the group consisting of —OMe, —OEt, —OPr, and —OBu. In further embodiments, R² is selected from the group consisting of —OCD₃, —OC₂D₅, —OC₃D₇, and —OC₄D₉. In yet further embodiments, R² is:

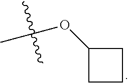 [SMILES: *OC1CCC1]

In still further embodiments, R² is:

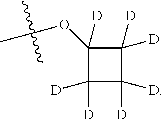

In certain embodiments, R³ is selected from the group consisting of methyl, ethyl, propyl, and butyl. In further embodiments, R³ is selected from the group consisting of CD₃, C₂D₅, C₃D₇, and C₄D₉. In yet further embodiments, R³ is:

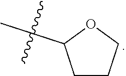 [SMILES: *C1CCCO1]

In still further embodiments, R³ is:

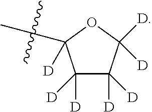

In certain embodiments, R³ is:

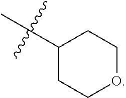 [SMILES: *C1CCOCC1]

In further embodiments, R³ is:

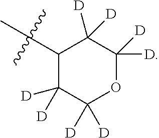

In yet further embodiments, R³ is:

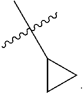 [SMILES: *C1CC1]

In still further embodiments, R³ is:

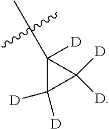

In certain embodiments, R³ is selected from the group consisting of —OMe, —OEt, —OPr, and —OBu. In further embodiments, R³ is selected from the group consisting of —OCD₃, —OC₂D₅, —OC₃D₇, and —OC₄D₉. In yet further embodiments, R³ is:

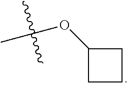 [SMILES: *OC1CCC1]

In still further embodiments, R³ is:

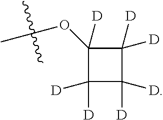

In certain embodiments, R⁴ is selected from the group consisting of methyl, ethyl, propyl, and butyl. In further embodiments, R⁴ is selected from the group consisting of CD₃, C₂D₅, C₃D₇, and C₄D₉. In yet further embodiments, R⁴ is:

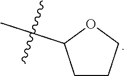 [SMILES: *C1CCCO1]

In still further embodiments, R⁴ is:

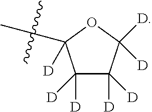

In certain embodiments, R⁴ is:

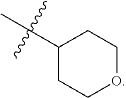 [SMILES: *C1CCOCC1]

In further embodiments, R⁴ is:

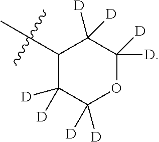

In yet further embodiments, R⁴ is:

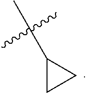 [SMILES: *C1CC1]

In still further embodiments, R⁴ is:

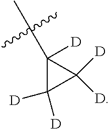

In certain embodiments, R⁴ is selected from the group consisting of —OMe, —OEt, —OPr, and —OBu. In further embodiments, R⁴ is selected from the group consisting of —OCD₃, —OC₂D₅, —OC₃D₇, and —OC₄D₉. In yet further embodiments, R⁴ is:

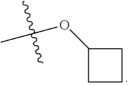 [SMILES: *OC1CCC1]

In still further embodiments, R⁴ is:

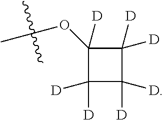

In certain embodiments, R⁵ is H. In further embodiments, R⁵ is halo. In yet further embodiments, R⁵ is F. In still further embodiments, R⁵ is selected from the group consisting of methyl, ethyl, propyl, and butyl. In certain embodiments, R⁵ is selected from the group consisting of CD₃, C₂D₅, C₃D₇, and C₄D₉. In further embodiments, R⁵ is:

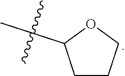 [SMILES: *C1CCCO1]

In yet further embodiments, R⁵ is:

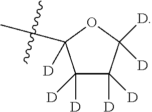

In still further embodiments, R⁵ is:

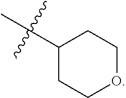 [SMILES: *C1CCOCC1]

In certain embodiments, R⁵ is:

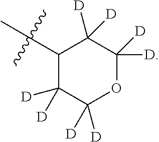

In further embodiments, R⁵ is:

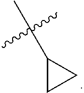 [SMILES: *C1CC1]

In yet further embodiments, R⁵ is:

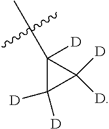

In certain embodiments, R⁵ is selected from the group consisting of —OMe, —OEt, —OPr, and —OBu. In further embodiments, R⁵ is selected from the group consisting of —OCD₃, —OC₂D₅, —OC₃D₇, and —OC₄D₉. In yet further embodiments, R⁵ is:

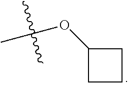 [SMILES: *OC1CCC1]

In still further embodiments, R⁵ is:

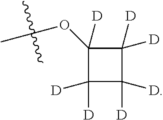

In certain embodiments, the compound is selected from the group consisting of:

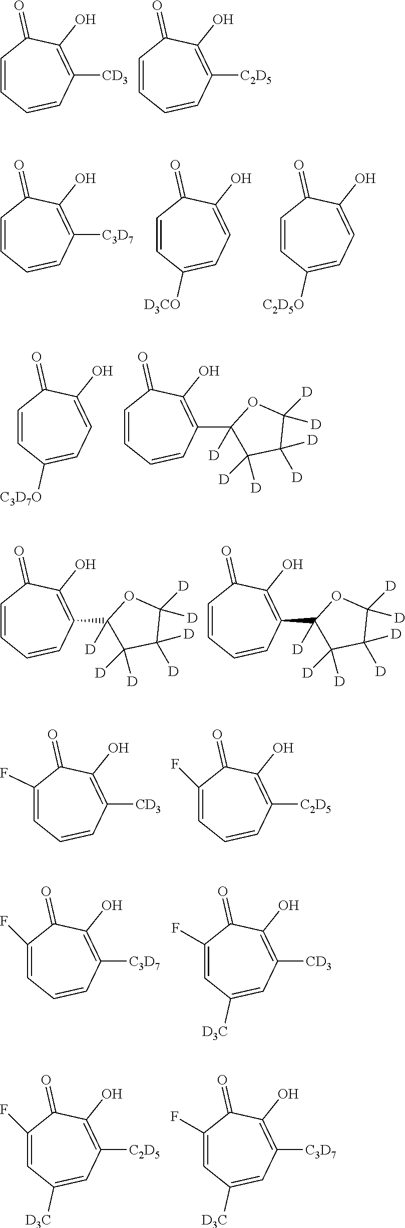

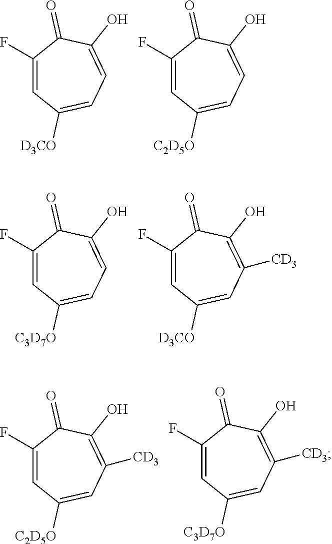

or a pharmaceutically acceptable salt thereof.

In further embodiments, the compound is selected from the group consisting of:

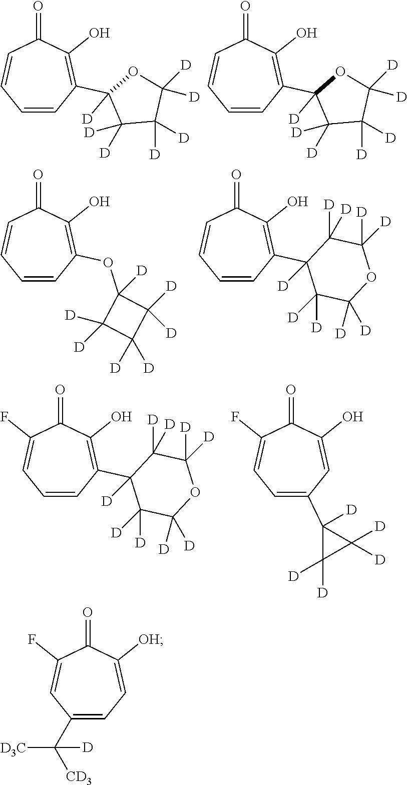

or a pharmaceutically acceptable salt thereof.

In certain embodiments, the compound is selected from the group consisting of:

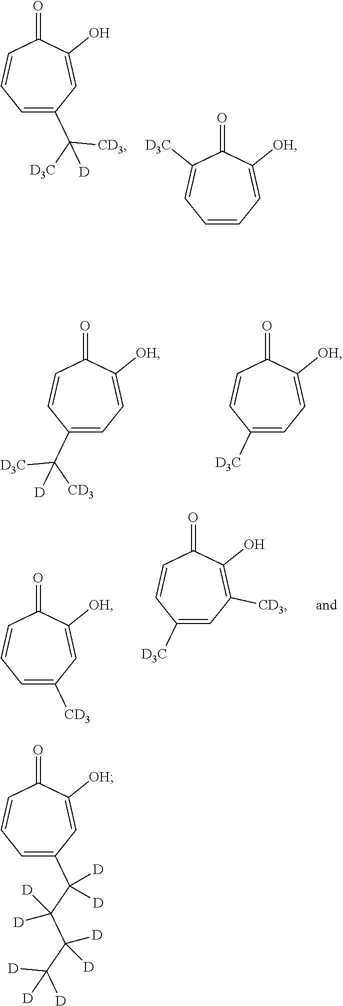

or a pharmaceutically acceptable salt thereof.

In further aspects, provided herein are pharmaceutical compositions, comprising a compound of the disclosure, or a pharmaceutically acceptable salt thereof; and a pharmaceutically acceptable carrier or excipient. In certain embodiments, the pharmaceutical compositions further comprise an additional therapeutic agent selected from the group consisting of an iron mobilizer and deferiprone.

### Methods of Treatment

In yet further aspects, provided herein are methods of treating a disease or condition characterized by decreased ferroportin, comprising administering to a subject in need thereof a therapeutically effective amount of a compound or pharmaceutical composition of the disclosure. In certain embodiments, the disease or condition characterized by decreased ferroportin is induced ferroportin disease associated with aging.

In still further aspects, provided herein are methods of treating a disease or condition characterized by decreased iron transport, comprising administering to a subject in need thereof a therapeutically effective amount of a compound or pharmaceutical composition of the disclosure.

In certain aspects, provided herein are methods of treating a disease or condition characterized by a defect or deficiency in an iron transporter, comprising administering to a subject in need thereof a therapeutically effective amount of a compound or pharmaceutical composition of the disclosure.

In further aspects, provided herein are methods of treating a disease or condition characterized by iron accumulation, comprising administering to a subject in need thereof a therapeutically effective amount of a compound or pharmaceutical composition of the disclosure. In certain embodiments, the iron accumulation is iron accumulation in the brain.

In yet further aspects, provided herein are methods of treating a disease or condition characterized by iron misdistribution, comprising administering to a subject in need thereof a therapeutically effective amount of a compound or pharmaceutical composition of the disclosure. In certain embodiments, the iron misdistribution is iron misdistribution in the brain.

In certain embodiments, the disease or condition is selected from the group consisting of anemia of inflammation, iron overload, thalassemia, hemochromatosis, atransferrinemia, myelodysplasia, hypochromic microcytic anemia, ferroportin disease, transfusional iron overload, and aceruloplasminemia.

In certain aspects, provided herein are methods of treating a neurodegenerative disorder, comprising administering to a subject in need thereof a therapeutically effective amount of a compound or pharmaceutical composition of the disclosure. In certain embodiments, the neurodegenerative disorder is selected from the group consisting of Parkinson's Disease, Alzheimer's Disease, Amyotrophic lateral sclerosis, Friedrich's ataxia, Huntington's Disease, Lewy Body Disease, and Spinal Muscular Atrophy. In further embodiments, the neurodegenerative disorder is neurodegeneration with brain iron accumulation (NBIA). In yet further embodiments, the NBIA is selected from the group consisting of Beta-propeller Protein-associated Neurodegeneration (BPAN), Pantothenate Kinase-associated Neurodegeneration (PKAN), PLA2G6-associated Neurodegeneration (PLAN), Mitochondrial-membrane Protein-associated Neurodegeneration (MPAN), Fatty Acid Hydroxylase-Associated Neurodegeneration (FAHN), COASY Protein-Associated Neurodegeneration (CoPAN), Aceruloplasminemia, Kufor-Rakeb Syndrome (also known as Parkinson's Disease 9 (PARK9)), Neuroferritinopathy, Woodhouse-Sakati Syndrome, and Idiopathic NBIA.

In certain embodiments, the methods of the disclosure further comprise administering to the subject an effective amount of an additional therapeutic agent selected from the group consisting of an iron mobilizer and deferiprone.

### EXAMPLES

The invention now being generally described, it will be more readily understood by reference to the following examples which are included merely for purposes of illustration of certain aspects and embodiments of the present invention, and are not intended to limit the invention.

### Example 1—Preparation of Boronic Acid Derivatives

To an oven-dried 40 mL vial equipped with a stir bar was added magnesium turnings (1 equiv.) after which the vial was evacuated 3× with N₂. To a second oven-dried 40 mL vial equipped with a stir bar under N₂ was added deuterated alkyl halide and dried Et₂O. The second vial was mixed briefly before the mixture was added dropwise to the Mg-containing vial. While the Grignard reagent was allowed to form over 2 hours, a third oven-dried vial equipped with a stir bar was placed under N₂ and trimethyl borate (1.2 equiv.) and dried Et₂O were added. The third vial was then cooled to −78 °C and the Grignard reagent was added dropwise over the course of 10 minutes. The solution was stirred for 20 minutes at −78 °C and after warmed to 23 °C over 1 hour. 1 M HCl solution was added, and the solution was allowed to stir for 30 minutes at 23 °C. Following, the reaction was extracted with Et₂O (2×6 mL), washed with brine (10 mL) and dried over Na₂SO₄. 0.5 mL of dried dioxane was added to the dried solution and ether was removed under a stream of N₂. The crude boronic acid in dioxane was utilized directly in the Suzuki-Miyuara coupling with the bromotropolone coupling partner.

### Example 2—Synthesis of Bromotropolone Scaffolds

### Alpha Bromotropolone Scaffold

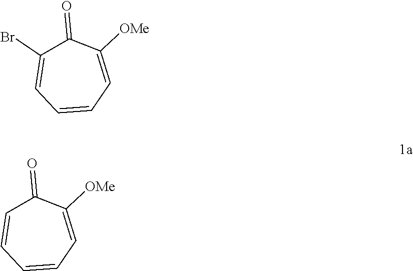 [SMILES: COc1ccccc(Br)c1=O ; COc1cccccc1=O]

An oven-dried two-neck round bottom flask was equipped with a condenser, evacuated, and backfilled 3× with N₂. Anhydrous K₂CO₃ (3 equiv.), Tropolone (1 equiv.), and 18-crown-6 (0.1 equiv.) were added, followed by MeCN. MeI was then added (5 equiv.), and the reaction was brought to reflux for 5 hours. The reaction was cooled and was then filtered through a pad of celite, rinsing with MeCN. The filtrate was then concentrated in vacuo and purified by flash column chromatography, yielding methyl-protected alpha bromotropolone 1a.

¹H NMR (500 MHz, CDCl₃) d 7.28-7.18 (m, 2H), 7.10 (ddd, J=10.8, 9.8, 1.0 Hz, 1H), 6.91-6.84 (m, 1H), 6.77 (dd, J=9.9, 0.8 Hz, 1H), 3.94 (s, 3H).

¹³C NMR (126 MHz, CDCl₃) 180.2, 165.1, 136.5, 136.5, 132.7, 127.8, 112.3, 56.1 HRMS (ESI+) Calc for C₈H₈O₂ [M+H]: 137.0603, Obs: 137.0603

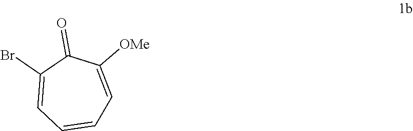 [SMILES: COc1ccccc(Br)c1=O]

To a 40 mL chem-vial with a stir bar, the previous product (1 equiv.) was added, followed by NBS (1.2 equiv.) (recrystallized from water), and CCl₄. The reaction was then heated at 90 °C for 3 hours. The reaction was then rinsed into a separatory funnel with DCM, washed with saturated sodium thiosulfate, then extracted 3× with DCM. The combined organic extracts were then washed with brine, dried over MgSO₄, then concentrated in vacuo. The product was then purified by flash chromatography yielding the alpha bromotropolone scaffold 1b.

¹H NMR (500 MHz, CDCl₃) d 8.19 (dd, J=9.5, 1.0 Hz, 1H), 7.15 (ddd, J=10.8, 9.9, 1.0 Hz, 1H), 6.79 (dt, J=9.9, 0.7 Hz, 1H), 6.68 (ddd, J=10.4, 9.5, 0.7 Hz, 1H), 3.96 (d, J=. 8 Hz, 3H)

¹³C NMR (126 MHz, CDCl₃) d 174.0, 162.9, 140.0, 138.0, 133.0, 125.4, 112.4, 56.9 HRMS (ESI+) Calc for C₈H702Br [M+H]: 214.9708, Obs: 214.9717

### Beta Bromotropolone Scaffold

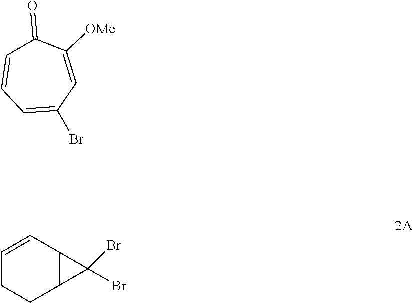 [SMILES: COc1cc(Br)cccc1=O ; BrC1(Br)C2C=CCCC21]

A large round bottom flask was equipped with an addition funnel, evacuated and backfilled with N2 3×. To this flask was added KOtBu (1.5 equiv.) and pentane. The solution was then submerged in an ice bath and cooled to 0 °C. Then, 1,3-cyclohexadiene was added via syringe (1 equiv.). CHBr3 (1.2 equiv.) was then added via the addition funnel over the course of 1 hour. The reaction was then left to stir at 0 °C for 1 hour, then warmed to room temperature and stirred for an additional 1 hour. Water was then added to the round bottom and stirred, then the reaction was transferred to a separatory funnel, extracted 3× with hexanes, dried with MgSO₄ and concentrated in vacuo. The product was then purified by flash chromatography, yielding desired intermediate 2a as an oil.

¹H NMR (500 MHz, CDCl₃) d 5.92 (app. d, J=2.9 Hz, 2H), 2.14 (dd, J=10.4, 2.2 Hz, 1H), 2.08-2.01 (m, 3H), 1.95-1.90 (m, 2H)

¹³C NMR (126 MHz, CDCl₃) δ 130.1, 122.2, 39.6, 29.6, 27.7, 21.0, 17.8 HRMS (ESI+) Calc for C₇H₈Br₂ [M+H]: 249.8993, Obs: 249.8996

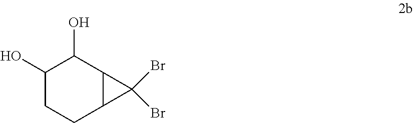 [SMILES: OC1CCC2C(C1O)C2(Br)Br]

To a 40 mL chem vial was added the 2a (1 equiv.), followed by a solution containing 1:1 tBuOH:H2O, NMO (2 equiv.), and freshly distilled pyridine (2.5 equiv.). Then, OsO4 was added (0.03 equiv.) as the amount from a 40 mg/mL solution in H₂O. The reaction was then heated to 100 °C and stirred vigorously for 3 hours. The reaction was then cooled and poured into an aqueous sodium thiosulfate solution and quenched overnight. The crude was then added to a separatory funnel and extracted 5× with Et2O. Organic extracts were washed with saturated aqueous CuSO₄ solution, water, and brine before being dried over MgSO₄ and concentrated in vacuo. The crude was then purified via flash column chromatography, yielding the desired product 2b as a brown solid.

¹H NMR (500 MHz, CD₃OD) d 3.77 (ddd, J=5.6, 3.0, 1.9 Hz, 1H), 3.5 (t, J=2.8 Hz, 1H), 2.12-2.03 (m, 1H), 2.03-1.96 (m, 1H), 1.85 (dd, J=11.1, 2.6 Hz, 1H), 1.70-1.54 (m, 2H), 1.41-1.26 (m, 1H).

¹³C NMR (126 MHz, CD₃OD) d 69.5, 67.8, 37.2, 34.2, 29.1, 26.6, 16.7 HRMS (ESI+) Calc for C₇H₁₀O₂Br₂ [M+Na]: 306.8945, Obs: 306.8948

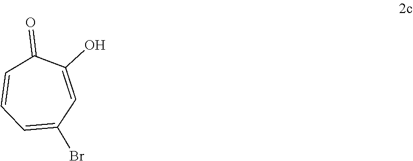

An oven-dried three-neck round bottom flask was equipped with a stir bar and placed under N₂. DCM (200 mL) and DMSO (4 equiv.) were added, and the reaction was cooled to −65 °C using an immersion cooler. After cooling, TFAA (4 equiv.) was added over 10 minutes via syringe. After 15 minutes, a solution of 2b (1 equiv.) was added in DMSO (10 mL) over 10 minutes. The reaction was allowed to proceed for 2 hours, then NEt3 (7 equiv.) was added over 10 minutes. The reaction was allowed to proceed for 2 hours, then was warmed to room temperature. The reaction was then quenched with 200 mL of 2M HCl, transferred to a separatory funnel, and extracted 3× with DCM. The combined organic layers were then washed three times with water, then once with brine, then dried over MgSO₄ and concentrated in vacuo. This mixture containing 2c was then subjected to the next step without purification.

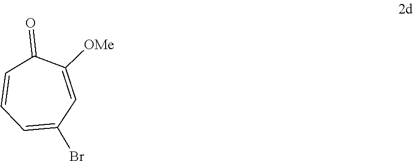 [SMILES: COc1cc(Br)cccc1=O]

An oven-dried two-neck round bottom flask was equipped with a condenser, evacuated, and backfilled 3× with N₂. Anhydrous K₂CO₃ (3 equiv.), mixture of 2c (1 equiv.), and 18-crown-6 (0.1 equiv.) were added, followed by MeCN. MeI was then added (5 equiv.), and the reaction was brought to reflux for 5 hours. The reaction was cooled and was then filtered through a pad of Celite, rinsing with MeCN. The filtrate was then concentrated in vacuo and purified by flash column chromatography, yielding methyl-protected beta bromotropolone.

¹H NMR (500 MHz, CDCl₃) d 7.63 (d, J=2.0 Hz, 1H), 7.06 (dd, J=11.4, 2.0 Hz, 1H), 6.79 (dd, J=11.4, 10.1 Hz, 1H), 6.59 (d, J=10.1 Hz, 1H), 3.90 (s, 3H)

¹³C NMR (126 MHz, CDCl₃) δ 177.2, 165.3, 139.3, 136.9, 132.5, 131.6, 111.0, 56.5 HRMS (ESI+) Calc for C₇H₅O₂Br [M+H]: 219.9708, Obs: 219.9704

### Gamma Bromotropolone Scaffold

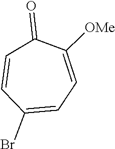 [SMILES: COc1ccc(Br)ccc1=O]

The Gamma bromotropolone Scaffold was synthesized following the same procedure as for the Beta Bromotropolone Scaffold while utilizing 1,4-cyclohexadiene in place of 1,3-cyclohexadiene in the first step and resulting in the following intermediates.

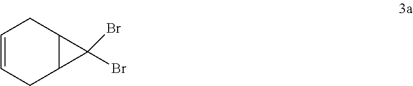

¹H NMR (500 MHz, CDCl₃) d 5.50 (s, 2H), 2.47 (d, J=17.5 Hz, 2H), 2.10 (d, J=17.5 Hz, 2H), 1.91 (dd, J=5.3, 2.4 Hz, 2H)

¹³C NMR (126 MHz, CDCl₃) d 122.6, 38.5, 25.1, 21.1

HRMS (ESI+) Calc for C₇H₈Br₂ [M+H]: 249.8993, Obs: 249.9005

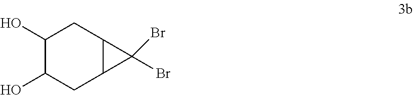 [SMILES: OC1CC2C(CC1O)C2(Br)Br]

¹H NMR (500 MHz, CD₃OD) d 3.66-3.59 (m, 2H), 2.17 (dtd, J=15.3, 6.6, 3.1 Hz, 1H), 1.99-1.90 (m, 2H), 1.76 (ddd, J=15.2, 4.7, 1.8 Hz, 2H)

¹³C NMR (126 MHz, CD₃OD) d 68.3, 40.2, 28.4, 28.0

HRMS (ESI+) Calc for C₇H₁₀O₂Br₂ [M+H]: 265.8942, Obs: 265.8941

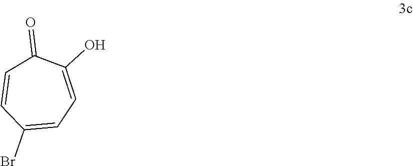

Used in next step without purification as above.

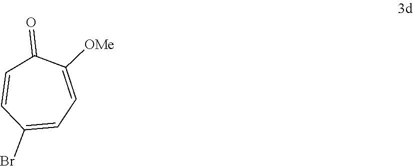 [SMILES: COc1ccc(Br)ccc1=O]

¹H NMR (500 MHz, CDCl₃) d 7.38 (dd, J=13.1, 2.0 Hz, 1H), 7.36 (dd, J=11.0, 2.0 Hz, 1H), 6.95 (d, J=13.0 Hz, 1H), 6.46 (d, J=10.9 Hz, 1H), 3.88 (s, 3H)

¹³C NMR (126 MHz, CDCl₃) δ 179.5, 164.8, 140.1, 136.1, 134.4, 122.6, 111.6, 56.5

HRMS (ESI+) Calc for C₇H₅O₂Br₂ [M+H]: 214.9708, Obs: 214.9710

### 1,3-Dibromotropolone Scaffold

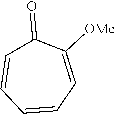

Synthesis is same as procedure listed for 1A

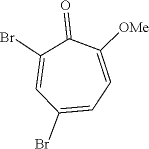 [SMILES: COc1ccc(Br)cc(Br)c1=O]

To an oven dried vial with stir bar was added 30 mL CCl₄ and methyl-protected tropolone from previous step (1 gram, 7.3 mmol). N-Bromosuccinimide (2.1 equiv.) was added in two portions before the vial was capped, sealed with electrical tape and heated to 90 C overnight. The reaction was then rinsed into a separatory funnel with DCM, washed with saturated sodium thiosulfate, then extracted 3× with DCM. The combined organic extracts were then washed with brine, dried over MgSO4, then concentrated in vacuo. The product was then purified by flash chromatography to yield the desired product.

¹H NMR (500 MHz, CDCl₃) δ 8.45 (d, 1H), 7.46 (d, 1H), 6.54 (d, 1H), 3.94 (s, 3H)

¹³C NMR (126 MHz, CDCl₃) δ 173.3, 161.9, 142.8, 137.0, 134.5, 119.5, 111.5, 56.9

### 1,3,5-Tribromotropolone Scaffold

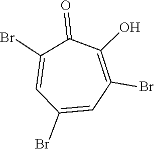 [SMILES: O=c1c(Br)cc(Br)cc(Br)c1O]

To an oven dried 40 mL vial with stir bar was added 30 mL CCl₄ and tropolone (1 gram, 8.1 mmol). N-Bromosuccinimide (3.2 equiv.) was added in three portions before the vial was capped, sealed with electrical tape and heated to 90 C overnight. The reaction was then rinsed into a separatory funnel with DCM, washed with saturated sodium thiosulfate, then extracted 3× with DCM. The combined organic extracts were then washed with brine, dried over MgSO₄, then concentrated in vacuo to yield pure product. The pure product was utilized directly in the next step.

¹H NMR (500 MHz, CDCl₃) δ 8.34 (s, 2H)

¹³C NMR (126 MHz, CDCl₃) δ 164.4, 144.0, 124.3, 118.0

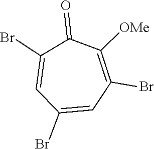 [SMILES: COc1c(Br)cc(Br)cc(Br)c1=O]

An oven-dried two-neck round bottom flask was equipped with a condenser, evacuated, and backfilled 3× with N₂. Anhydrous K₂CO₃ (16.97 g, 123 mmol, 3 equiv.), 7a (5 g, 41 mmol, 1 equiv.), and 18-crown-6 (1.08 g, 4.1 mmol, 0.1 equiv.) were added, followed by MeCN (250 mL). MeI was then added (29.1 g, 205 mmol, 5 equiv.), and the reaction was brought to reflux for 3 hours. The reaction was cooled and was then filtered through a pad of celite, rinsing with acetone. The filtrate was then concentrated in vacuo and purified by flash column chromatography, yielding product as a yellow solid.

¹H NMR (500 MHz, CDCl₃) δ 8.28 (s, 1H), 7.95 (s, 1H), 4.01 (s, 3H)

### Example 3—General Procedure for Derivatives Containing α′-Fluorine/α-Substitution

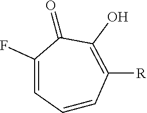

Benzyl-protected α-Bromotropolone scaffold was prepared as previously described (4). To a mixture of this scaffold in DMSO was added dried CsF (1.5 equiv.) under N₂ atmosphere. The mixture was stirred at 110 °C under N₂ for 8 hours. The reaction was cooled to 25 °C and brine was added. The aqueous mixture was extracted with EtOAc, combined organic layer was washed with brine and then dried over Na₂SO₄. The solution was concentrated and purified via silica gel chromatography yielding product.

The acquired residue was then deprotected as previously described using TFA at 50 °C to yield the α-Fluorotropolone scaffold. Briefly, the residue was dissolved in TFA and the mixture was stirred for 1 hour at 50 °C. The crude product after the mixture was concentrated under vacuum was utilized in the next step.

To a solution of the crude product in CCl₄ was slowly added NBS (1.3 equiv.) at 25 °C under N₂. The mixture was heated to 80 °C for 3 hours. After, the mixture was cooled to 25 °C and water was added. The aqueous mixture was extracted with 3× dichloromethane and the combined organics were washed with brine and dried over Na₂SO₄. Following filtration, the solution was concentrated to dryness giving the fluoro/bromo intermediate as a crude mixture which was utilized in the next step without purification.

The intermediate was then reprotected utilizing methyl protection procedure as previously described (4). Briefly, the crude residue was resuspended in MeCN to which K₂CO₃ (3 equivs.) and 18-crown-6 (0.1 equivs.) were added under N₂. MeI (4 equivs.) was then added dropwise and the reaction was heated to 85 °C and allowed to run for 12 hours. Following cooling, the mixture was filtered and the residue concentrated under reduced pressure to yield crude product. The crude product was purified via silica gel chromatography to yield the desired methylated fluoro/bromotropolone scaffold. This scaffold was then utilized in GP1 followed by GP2 to give desired α-fluoro/α-substituted derivatives.

### Example 4—General Procedure for Derivatives Containing α′-Fluorine/α,γ-Substitution

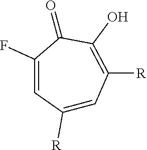

Methyl-protected α-Fluoro/α-substituted derivatives were acquired as depicted above after GP1. To a solution of this product in CHCl₃ was slowly added NBS (1.3 equiv.) at 25 °C under N₂. The mixture was heated to 80 °C for 3 hours. After, the mixture was cooled to 25 °C and water was added. The aqueous mixture was extracted with 3× dichloromethane and the combined organics were washed with brine and dried over Na₂SO₄. Following filtration, the solution was concentrated to dryness giving the α-Fluorine/α-substituted/γ-Bromo intermediate which was purified via silica gel chromatography. The purified product was utilized in GP1 followed by GP2 to yield α-Fluorine/α,γ-substituted derivatives.

### Example 5—General Procedure for Derivatives Containing α′-Fluorine/γ-Substitution

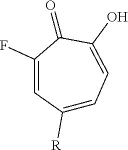

The γ-Bromotropolone scaffold was synthesized as described in Example 2. This scaffold was then utilized in GP1 to acquire the protected coupled product. Following, this product was redissolved in CHCl₃ and NBS (1.3 equiv.) was slowly added at 25 °C under N₂. The mixture was heated to 80 °C for 3 hours. After, the mixture was cooled to 25 °C and water was added. The aqueous mixture was extracted with 3× dichloromethane and the combined organics were washed with brine and dried over Na₂SO₄. Following filtration, the solution was concentrated to dryness giving the α′-Bromo/γ-substituted intermediate which was purified via silica gel chromatography. The purified product was utilized in the α′-fluorination procedure as previously described.

Briefly, to a mixture of this scaffold in DMSO was added dried CsF (1.5 equiv.) under N₂ atmosphere. The mixture was stirred at 110 °C under N₂ for 8 hours. The reaction was cooled to 25 °C and brine was added. The aqueous mixture was extracted with EtOAc, combined organic layer was washed with brine and then dried over Na₂SO₄. The solution was concentrated and purified via silica gel chromatography yielding protected product. This product was then deprotected utilizing GP2 to yield the desired final product.

### Example 6—Procedure for α-Per-Deuterated Furan Derivative

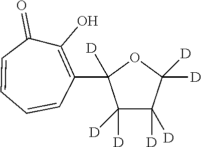 [SMILES: [2H]C1([2H])OC([2H])(c2ccccc(=O)c2O)C([2H])([2H])C1([2H])[2H]]

The Benzyl-protected α-Bromotropolone scaffold, which was utilized in the Heck coupling, was prepared as described in Example 2.

Preparation of the 2′3′-dideuterofuran-d₆ (a) (Based on synthesis from J. Org. Chem. 2007, 72, 19, 7253-7259)

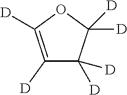 [SMILES: [2H]C1=C([2H])C([2H])([2H])C([2H])([2H])O1]

LiAlD₄ was suspended in THF and refluxed for 30 minutes. Upon cooling to −55 °C utilizing an immersion cooler, a solution of Succinic anhydride-d₄ in THF was added dropwise over 30 minutes. The solution was warmed to 25 °C over 90 minutes and then cooled to −15 °C for 15 minutes. 6M HCl solution was added slowly and the solution was warmed to 25 °C and stirred for 20 minutes. The reaction mixture was washed with brine and the organics extracted with 3×Et₂O. the combined organics were dried over Na₂SO₄ and concentrated under vacuum. The residue was purified via fractional distillation (BP 65 °C) to yield the desired product.

This product was then dissolved in dichloromethane and cooled to −78 °C using immersion cooler for 2 hours. A solution of cold DIBAL-D in DCM (4 °C) was added slowly and the reaction was allowed to proceed for 2 hours. The reaction was quenched with slow addition of MeOD and sat. Rochelle's Salt and the reaction was allowed to warm to 25 °C where a precipitate formed. The solid was resuspended in dichloromethane and filtrate was concentrated to yield the crude lactol, which was used immediately in the next step without purification

A three-necked flask was charged solution of TsOH in quinoline and a distillation apparatus was assembled, with a 2M aqueous solution of NaOH kept frozen around the bottom of the receiver flask. The quinoline solution was submerged in a 190 °C wax bath and the receiver flask was maintained at −78 °C in a dry ice/acetone bath. The crude product from the previous step was added dropwise into the quinoline solution and the solution was heated to 210 °C for an hour. The recovered liquid was removed from the apparatus quickly and purified via distillation (BP 54 °C) to yield the final desired product.

2′3′-dideuterofuran-d6 (a) and Benzyl-protected α-Bromotropolone scaffold were utilized in a Heck coupling as previously described in Example 2.

Briefly, to a solution of a (5 equivs.) and the benzyl-protected α-Bromotropolone scaffold in dioxane in a pressure flask was added K₂CO₃ (2 equivs.), Ph₃P (0.20 equivs.) and Pd(OAc)₂ (0.10 equivs.) in a glove box under Ar. The mixture was then heated to 110 °C for 2 hours, after which the reaction was cooled and water was added. The mixture was extracted with 2×EtOAc, the combined organics were washed with brine and dried over Na₂SO₄. The crude product was concentrated under vacuum and purified via silica gel chromatography to yield the racemic Heck-coupled intermediate (b).

To a solution of the intermediate (b) in MeOD was added Rh(PPh₃)₃Cl under N₂. The reaction flask was evacuated and refilled with D₂ 3× and the mixture was stirred under D₂ (1 atm) for 4 hours. The reaction mixture was filtered through Celite, rinsing with MeOH and then concentrated under vacuum to give the desired product as a racemic mixture. The mixture was resolved using chiral SFC with a DAICEL CHIRALPAK AD (250 mm*30 mm, 10 mm) as the stationary phase. The separated enantiomers were then employed in the final step separately to acquire the desired products.

The enantiomers were separately dissolved in neat TFA and heated to 50 °C for 2 hours with stirring. The reaction mixtures were cooled and then concentrated under vacuum to give the crude products. The residues were purified by preparatory HPLC separately, employing a Nano-micro Kromasil C18 (100 mm*30 mm, 8 mm) as the stationary phase, to yield the final desired products as pure enantiomers.

### Example 7—General Procedure for Derivative Preparation Via Csp2-Csp3 Bond Formation

In general, per-deuterated derivatives were synthesized as follows: Bromotropolone scaffolds were synthesized as described above. An Oven-dried 8 mL vial equipped with a stir bar and the vial containing the crude boronic acid were carried into a glove box filled with Ar. In the glovebox, all solid components were first added: Bromotropolone scaffold (0.5 mmol, 1 equiv.), Pd₂(dba)₃ (0.05 mmol, 0.1 equiv.), P(o-tol)₃ (0.1 mmol, 0.2 equiv.), Ag₂O (0.75 mmol, 1.5 equiv.). Then, an additional 0.5 mL of dried dioxane was added to the crude boronic acid mixture (1 mL total dioxane) before addition of the boronic acid in dioxane to the chemvial using a micropipettor. Multibrominated scaffolds utilized reagent equivalents per bromide such that, for example, for the tribromotropolone scaffold, Pd₂(dba)₃ (0.15 mmol, 0.3 equiv.), P(o-tol)₃ (0.3 mmol, 0.6 equiv.), Ag₂O (2.25 mmol, 4.5 equiv.) were used in 3 mL total of dried dioxane. For the dibromotropolone scaffold, Pd₂(dba)₃ (0.1 mmol, 0.2 equiv.), P(o-tol)₃ (0.2 mmol, 0.4 equiv.), Ag₂O (1.5 mmol, 3 equiv.) were used in 2 mL total of dried dioxane. The vial was then capped and removed from the glove box and stirred at 85 °C for 36 hours. After this, the crude reaction was passed through a silica plug, rinsing with EtOAc, then concentrated in vacuo. The mixture was then dissolved in HPLC grade MeOH (5 mL) before purification by preparative HPLC on an Agilent 1200 series HPLC equipped with a Waters Sunfire OBD Prep Column (100 Å, 5 μm, 30 mm×150 mm), employing MeCN and H₂O as the mobile phases, 40 mL/min flow rate, monitoring at 238 nm. Purified material was collected, then concentrated in vacuo, using additional MeCN to azeotrope off remaining water as needed.

An alternative method for forming C—C bonds between sp2 and sp3 centers involves the use of Grignard reagents to produce Kumada couplings starting with haloalkane counterparts (J. Organomet. Chem. 1997, 532, 1, 271-273; Bioorg. Med. Chem. 2007, 15, 22, 7144-7165; Adv. Synth. Catal. 2019, 361, 19, 4468-4473). 2-hydroxy-4-(isopropyl-d₇)-cyclohepta-2,4,6-trien-1-one and 2-hydroxy-5-(isopropyl-d₇)-cyclohepta-2,4,6-trien-1-one could be made utilizing perdeuterated haloalkanes in similar fashion.

### Acid Deprotection of Methyl Ether Coupled Products.

An 8 mL chemvial was equipped with a stir bar. The purified material from GP1 was dissolved in dioxane (0.11 M) and added to the vial. Next, a solution of 20% H₂SO₄ was added (0.11 M, equal volume to dioxane). This was then heated to 100 °C and stirred for 3 hours. After this, the reaction mixture was diluted with water and extracted 3× with ether. The combined organic extracts were then dried over Na₂SO₄ and concentrated in vacuo to yield final products.

### Example 8-Characterization Data for Select Compounds

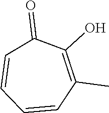

### 2-hydroxy-3-methylcyclohepta-2,4,6-trien-1-one

¹H NMR (500 MHz, CDCl₃) δ 7.55-7.51 (m, 1H), 7.35 (dd, J=10.3, 1.2 Hz, 1H), 7.32-7.27 (m, 1H), 6.97 (td, J=9.8, 1.2 Hz, 1H), 2.48 (s, 3H).

¹³C NMR (126 MHz, CDCl₃) δ 172.5, 167.6, 139.9, 138.1, 135.8, 127.4, 121.2, 22.2.

HRMS (ESI+) Calc for C₈H₈O₂ [M+H]: 137.0603, Obs: 137.0602

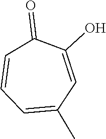

### 2-hydroxy-4-methylcyclohepta-2,4,6-trien-1-one

¹H NMR (500 MHz, CDCl₃) δ 7.30-7.24 (m, 3H), 6.92 (m, 1H), 2.47 (s, 3H)

¹³C NMR (126 MHz, CDCl₃) δ 170.8, 170.7, 149.9, 136.7, 129.7, 125.7, 121.9, 27.2

HRMS (ESI+) Calc for C₈H₈O₂ [M+H]: 137.0603, Obs: 137.0607

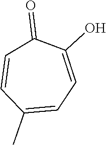

### 2-hydroxy-5-methylcyclohepta-2,4,6-trien-1-one

¹H NMR (500 MHz, CDCl₃) δ 7.32-7.26 (m, 4H), 2.45 (s, 3H)

¹³C NMR (126 MHz, CDCl₃) δ 170.7, 138.4, 123.9, 25.9

HRMS (ESI+) Calc for C₈H₈O₂ [M+H]: 137.0603, Obs: 137.0600

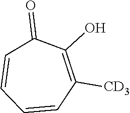 [SMILES: [2H]C([2H])([2H])c1ccccc(=O)c1O]

### 2-hydroxy-3-(methyl-d₃)cyclohepta-2,4,6-trien-1-one

¹H NMR (500 MHz, CDCl₃) δ 7.52 (d, 1H), 7.35 (d, 1H), 7.29 (t, 1H), 6.97 (t, 1H)

²H NMR (500 MHz, CDCl₃) δ 2.45

¹³C NMR (126 MHz, CDCl₃) δ 172.5, 167.5, 140.5, 137.8, 135.7, 127.3, 121.2

HRMS (ESI+) Calc for C₈H₅D₃O₂ [M+H]: 140.0791, Obs: 140.0793

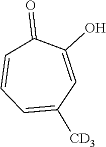 [SMILES: [2H]C([2H])([2H])c1cccc(=O)c(O)c1]

### 2-hydroxy-4-(methyl-d₃)cyclohepta-2,4,6-trien-1-one

¹H NMR (500 MHz, CDCl₃) δ 7.30-7.24 (m, 3H), 6.92 (m, 1H)

²H NMR (500 MHz, CDCl₃) δ 2.46

¹³C NMR (126 MHz, CDCl₃) δ 170.8, 170.7, 149.9, 136.8, 129.8, 125.7, 122.0

HRMS (ESI+) Calc for C₈H₅D₃O₂ [M+H]: 140.0791, Obs: 140.0796

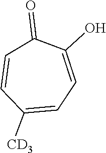 [SMILES: [2H]C([2H])([2H])c1ccc(O)c(=O)cc1]

### 2-hydroxy-5-(methyl-d₃)cyclohepta-2,4,6-trien-1-one

¹H NMR (500 MHz, CDCl₃) δ 7.32-7.26 (m, 4H)

²H NMR (500 MHz, CDCl₃) δ 2.39

¹³C NMR (126 MHz, CDCl₃) δ 170.7, 138.5, 124.0

HRMS (ESI+) Calc for C₈H₅D₃O₂ [M+H]: 140.0791, Obs: 140.0794

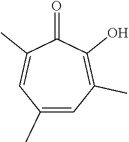

### 2-hydroxy-1,3,5-trimethylcyclohepta-2,4,6-trien-1-one

¹H NMR (500 MHz, CD₃CN) δ 7.39 (s, 2H), 2.38 (s, 3H), 2.37 (s, 6H)

¹³C NMR (126 MHz, CD₃CN) δ 167.8, 140.1, 136.8, 135.0, 26.2, 21.5

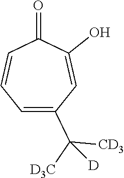 [SMILES: [2H]C([2H])([2H])C([2H])(c1cccc(=O)c(O)c1)C([2H])([2H])[2H]]

### 2-hydroxy-4-(isopropyl-d₇)-cyclohepta-2,4,6-trien-1-one

¹H NMR (500 MHz, CDCl₃) δ 7.30-7.24 (m, 3H), 6.95 (m, 1H)

²H NMR (500 MHz, CDCl₃) δ 2.90, 1.27

¹³C NMR (126 MHz, CDCl₃) δ 170.8, 170.7, 149.9, 136.5, 129.7, 125.6, 121.5,

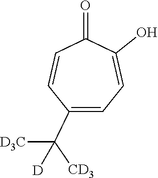 [SMILES: [2H]C([2H])([2H])C([2H])(c1ccc(O)c(=O)cc1)C([2H])([2H])[2H]]

### 2-hydroxy-5-(isopropyl-d₇)-cyclohepta-2,4,6-trien-1-one

¹H NMR (500 MHz, CDCl₃) δ 7.32-7.26 (m, 4H)

²H NMR (500 MHz, CDCl₃) δ 2.92, 1.26

¹³C NMR (126 MHz, CDCl₃) δ 170.7, 144.0, 138.2, 124.1

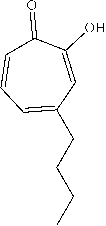

### 2-hydroxy-4-butylcyclohepta-2,4,6-trien-1-one

¹H NMR (500 MHz, CDCl₃) δ 7.30-7.24 (m, 3H), 6.92 (dd, 1H), 2.63 (dd, 2H), 1.64 (m, 2H), 1.38 (tq, 1H), 0.94 (t, 1H)

¹³C NMR (126 MHz, CDCl₃) δ 170.9, 170.5, 154.6, 136.9, 129.4, 125.2, 122.1, 41.0, 33.4, 22.2, 13.8

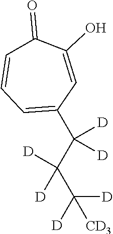 [SMILES: [2H]C([2H])([2H])C([2H])([2H])C([2H])([2H])C([2H])([2H])c1cccc(=O)c(O)c1]

### 2-hydroxy-4-(butyl-d₉)cyclohepta-2,4,6-trien-1-one

¹H NMR (500 MHz, CDCl₃) δ 7.30-7.24 (m, 3H), 6.92 (m, 1H)

²H NMR (500 MHz, CDCl₃) δ 2.57, 1.56, 1.30, 0.87

¹³C NMR (126 MHz, CDCl₃) δ 170.9, 170.7, 154.6, 136.9, 129.4, 125.2, 122.1

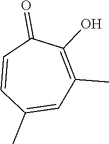

### 2-hydroxy-1,3-dimethylcyclohepta-2,4,6-trien-1-one

¹H NMR (500 MHz, CDCl₃) δ 7.43 (s, 1H), 7.26 (d, 1H), 7.16 (d, 1H), 2.40 (s, 3H), 2.37 (s, 3H)

¹³C NMR (126 MHz, CDCl₃) δ 171.9, 166.4, 141.3, 137.7, 137.5, 136.0, 121.4, 26.0, 22.3

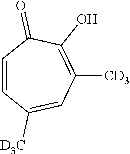 [SMILES: [2H]C([2H])([2H])c1ccc(=O)c(O)c(C([2H])([2H])[2H])c1]

### 2-hydroxy-1,3-(dimethyl-d₆)cyclohepta-2,4,6-trien-1-one

¹H NMR (500 MHz, CDCl₃) δ 7.43 (s, 1H), 7.26 (d, 1H), 7.16 (d, 1H)

²H NMR (500 MHz, CDCl₃) δ 2.41, 2.37

¹³C NMR (126 MHz, CDCl₃) δ 171.9, 166.2, 141.4, 137.7, 137.5, 136.0, 121.3

### Example 9—Preparation of Liposomes

Liposomes were prepared as previously described (A. S. Grillo et al. Science, 356, 608-615 (2017)). Lipid films were generated through addition of 200 μL of 4 mg/mL recrystallized cholesterol in chloroform to a 960 μL solution of 25 mg/mL POPC in chloroform (Avanti Polar Lipids 850457C). After evaporation under a stream of nitrogen, films were placed under hi-vacuum for 18 hours to ensure quantitative solvent removal. Lipid films were rehydrated by adding 1 mL of liposome inside buffer (15 mM FeCl₃, 125 mM citrate, 50 mM MES/TRIS pH 7.0) to the lipid film and thoroughly vortexing to form a solution of multilamellar vesicles (MLVs). Liposomes were then extruded through an Avanti Lipids extruder (Avanti Polar Lipids 610023) using a 0.2 mm membrane (Whatman 800281), passing the MLV suspension through the filter 21 times to ensure homogenous large unilamellar vesicles (LUVs). LUVs were subjected to size exclusion chromatography, using a ~20 cm column of Sephadex® G-50 Medium resin that was hydrated in liposome outside buffer (600 mM sodium ascorbate, 50 mM MES/TRIS pH 7.0). Separation of LUVs from extravesicular Fe could be readily visualized on the column, and the most concentrated portion of LUVs was collected.

### Example 10—Quantification of Liposomes

Determination of total phosphorous was performed utilizing the Bartlett Assay. Briefly, the LUV suspension was diluted 10-fold in liposome outside buffer, then 10 μL of this solution was placed in three 7 mL vials along with 10 μL of liposome outside buffer for background. 450 mL of 8.9 M H2SO₄ was then added, and the mixture was heated open to air at 225 °C for 25 minutes. Following cooling, 150 mL of 30% H₂O₂ was added, and the mixture was further heated open to air at 225 °C for 30 min. The samples were then allowed to fully cool and diluted using 3.9 mL of Milli-Q water. 500 mL of 2.5% w/v ammonium molybdate was then added, then the samples were vortexed, then 500 mL of 10% w/v ascorbic acid was added. The samples were then re-vortexed, capped, and heated at 100 °C for seven minutes. The samples were allowed to fully cool then read at A820 and compared to a phosphorous standard curve to determine the total quantity of phosphorous.

### Example 11—Liposome Efflux Assay

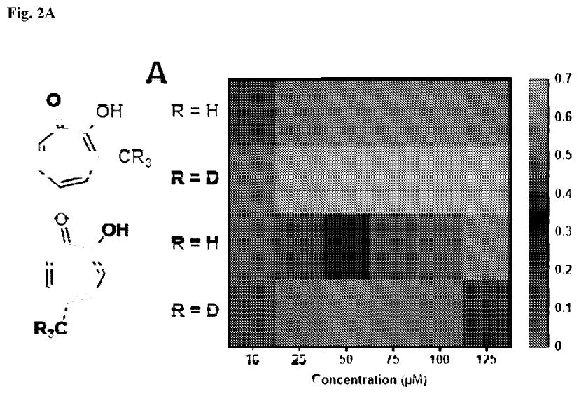

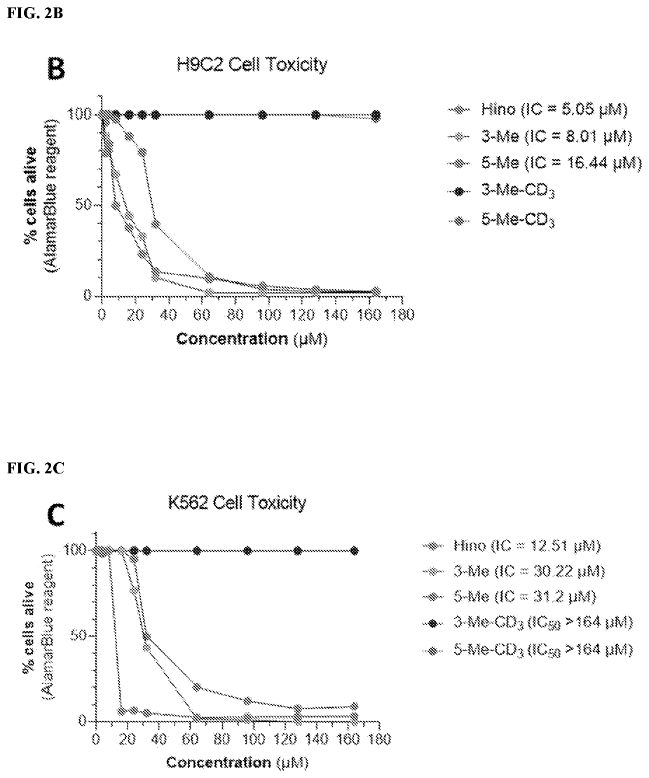

Data for this experiment are shown in FIG. 2A.

16 mL of liposome suspension was created with the following components: 160 mL of ferrozine solution (100× stock, 50 mM in liposome outside buffer), LUV suspension (to a final concentration of 1 mM phosphorous), and liposome outside buffer up to 16 mL. 195 mL of liposome suspension was then added to a 96 well plate (NUM) using a multichannel pipettor. The plate was then placed in a plate reader and equilibrated at 37 °C for 5 minutes, then read for a baseline measurement. Following this equilibration, compound was added at the designated concentrations (40× stock in DMSO) using a multichannel pipettor, and the plate was then incubated at 37 °C for 40 min. Reads were taken every minute at A562 and the plate mixed at 220 rpm. Total iron efflux was determined via ferrocene-iron complex signal. These experiments were performed in triplicate, with a minimum of two biological replicates.

### Example 12—H9C2 Toxicity Assay

Data for this experiment are shown in FIG. 2B.

H9C2 Cells (ATCC CRL-1446) were cultured at 37 °C/5% CO₂ in DMEM media supplemented with 10% FBS (Gemini 900-108), 1% Penicillin Streptomycin (Gibco 15140-122) and 1% MEM Non-Essential Amino Acids (Corning 25-025-Cl) in T175 culture flasks (Thermofisher) until at least passage 5. Through centrifugation and dilution of cellular pellet, a 5×10⁵ cells/mL solution was obtained. A 96-well compound plate containing 40×DMSO Solutions of desired test compounds over desired concentration range was created using 100 mM DMSO stock solutions. Using a multichannel micropipetor, 3.34 μL of each stock solution was added to 297 μL of cell media in a 96 well plate with light mixing via pipetting up and down. To a 96 well tissue culture plate, 3×80 μL of each created compound solution was added to successive rows, with the top and bottom rows filled with cell media to reduce evaporation. Following, 20 μL of the 5×10⁵ cell/mL solution was added to each well using a micropipettor so that each well contained 1×10⁴ cells. Plates were incubated at 37 °C/5% CO₂ for four days before AlamarBlue reagent was utilized to determine cell viability in accordance with assay protocol. Briefly, 10 μL of AlamarBlue reagent was added to each well and the plates were then incubated for 3 hours. Cell viability was determined via fluorescent signal (Em555/Ex585) using a microplate reader (BioTek Synergy H1 Hybrid Reader) and comparison to Puromycin and DMSO-treated wells as positive and negative controls, respectively.

### Example 13—K562 Toxicity Assay

Data for this experiment are shown in FIG. 2C.

K562 Cells (ATCC CCL-243) were cultured at 37 °C/5% CO₂ in IMDM (Iscove's Modified Dulbeco's Medium, ATCC 30-2005) media supplemented with 10% FBS (Gemini 900-108), 1% Penicillin Streptomycin (Gibco 15140-122) and 1% MEM Non-Essential Amino Acids (Corning 25-025-Cl) in T175 culture flasks (Thermofisher) until at least passage 5. Through centrifugation and dilution of cellular pellet, a 1.25×10⁶ cells/mL solution was obtained. A 96-well compound plate containing 40×DMSO Solutions of desired test compounds over desired concentration range was created using 100 mM DMSO stock solutions. Using a multichannel micropipetor, 3.34 μL of each stock solution was added to 297 μL of cell media in a 96 well plate with light mixing via pipetting up and down. To a 96 well plate, 3×80 μL of each created compound solution was added to successive rows, with the top and bottom rows filled with cell media to reduce evaporation. Following, 20 μL of the 1.25×10⁶ cell/mL solution was added to each well using a micropipettor so that each well contained 2.5×10⁴ cells. Plates were incubated at 37 °C/5% CO₂ for four days before AlamarBlue reagent was utilized to determine cell viability in accordance with assay protocol. Briefly, 10 μL of AlamarBlue reagent was added to each well and the plates were then incubated for 3 hours. Cell viability was determined via fluorescent signal (Em555/Ex585) using a microplate reader (BioTek Synergy H1 Hybrid Reader) and comparison to Puromycin and DMSO-treated wells as positive and negative controls, respectively.

### Example 14—Procedure for C. elegans Experiments

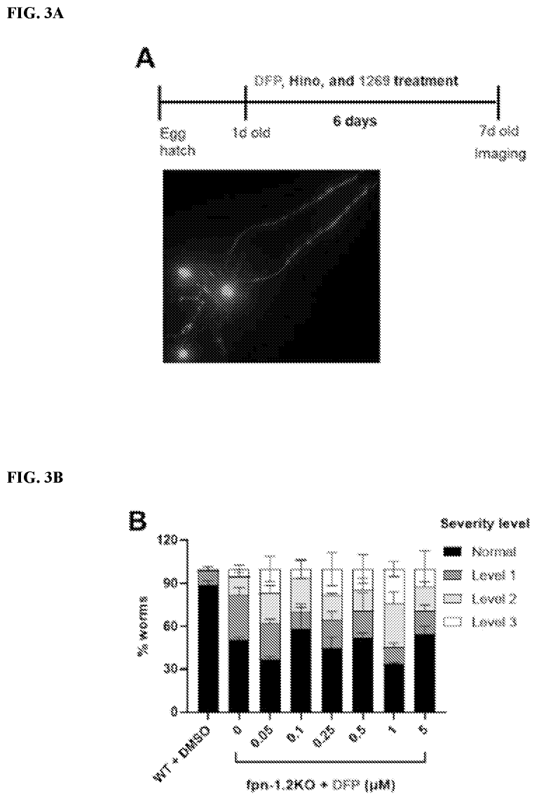

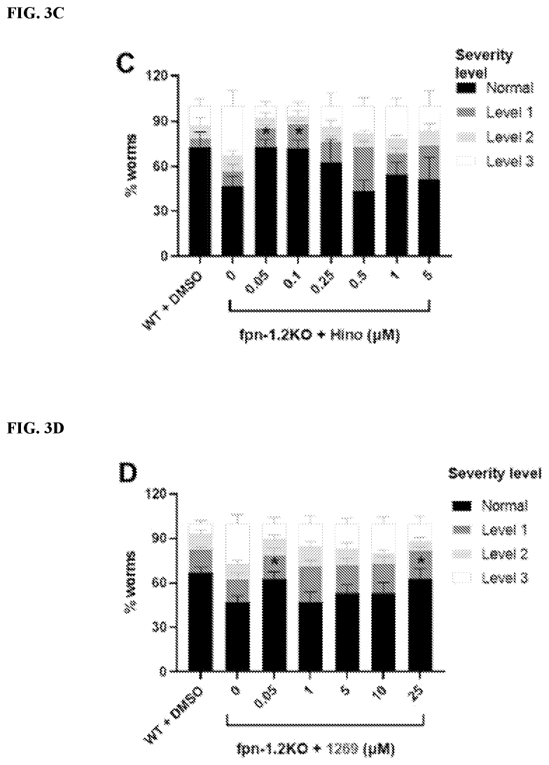

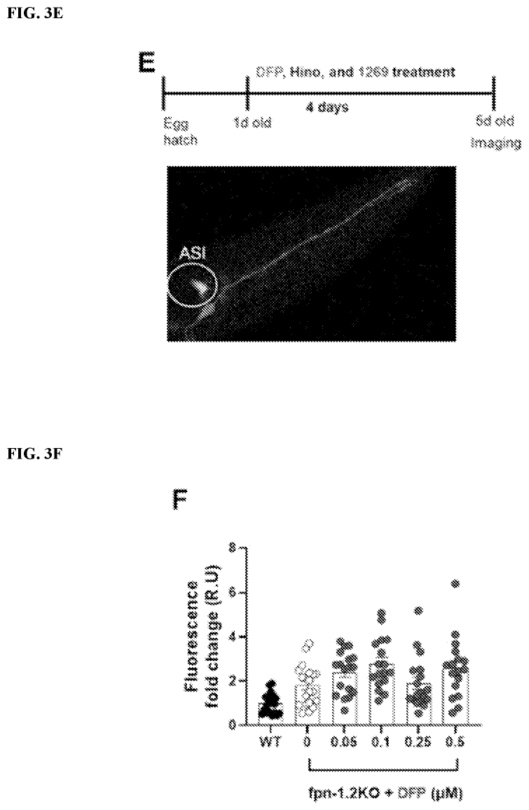

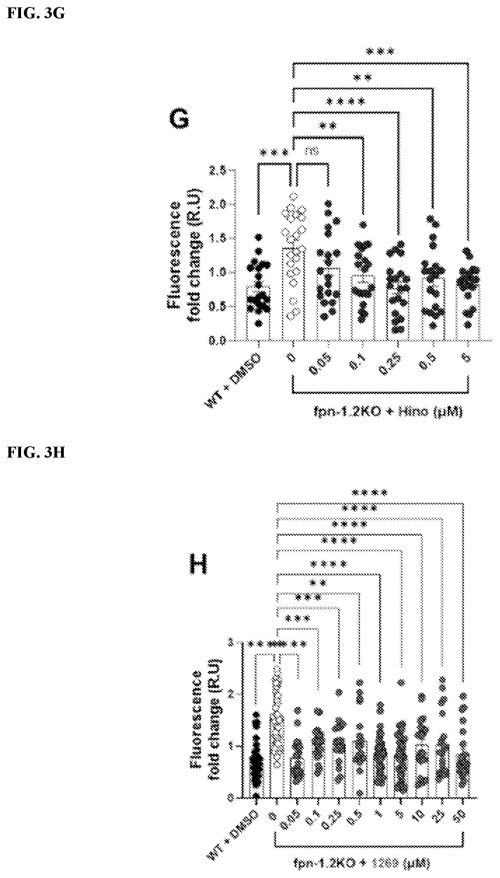

Data for this experiment are shown in FIG. 3A-3H.

C. elegans strains:
BY200 vtIs1[pdat-1::gfp; rol-6] V
NES277 fpn-1.2-KO V; vtIs1[pdat-1::gfp; rol-6] V
GA631 wuIs177 [ftn-1p::GFP+lin-15(+)]X
NES281 fpn-1.2-KO V; wuIs177 [ftn-1p::gfp+lin-15(+)]X

### Analysis of Dopaminergic Neurodegeneration

BY200 and NES277 strains were grown in normal NGM plates. At age day 1, worms were transferred to NGM plates containing vehicle (DMSO) or various concentrations of deferiprone (DFP), hinokitiol (Hino), and FeM-1269. Worms were passaged daily until being used for imaging at day 7 using Zeiss AxioImager microscope equipped with DIC and fluorescence. Data analysis was done blinded according to a previously described protocol (R. Nass, D. H. Hall, D. M. Miller, R. D. Blakely, Neurotoxin-induced degeneration of dopamine neurons in Caenorhabditis elegans. Proceedings of the National Academy of Sciences 99, 3264-3269 (2002).).

### Measurement of Ferritin Fluorescence

GA631 and NES281 strains were grown in normal NGM plates. At age day 1, the worms were transferred to NGM plates containing vehicle (DMSO) or various concentrations of DFP, Hino, and FeM-1269. Worms were passaged daily until being used for imaging at day 5 using Zeiss LSM880 at 20× magnification with focus on the head. Fluorescence intensity was quantified blinded using ImageJ.

### Example 15—Procedure for Brain Iron Level Measurements in Flatiron Mice

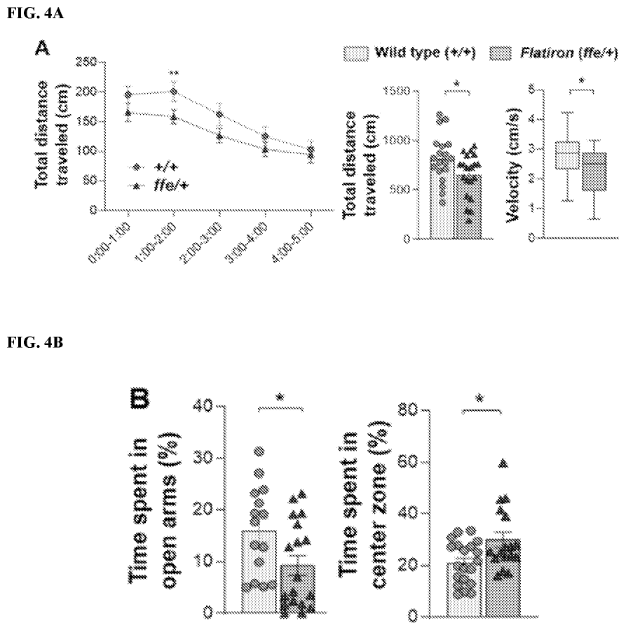

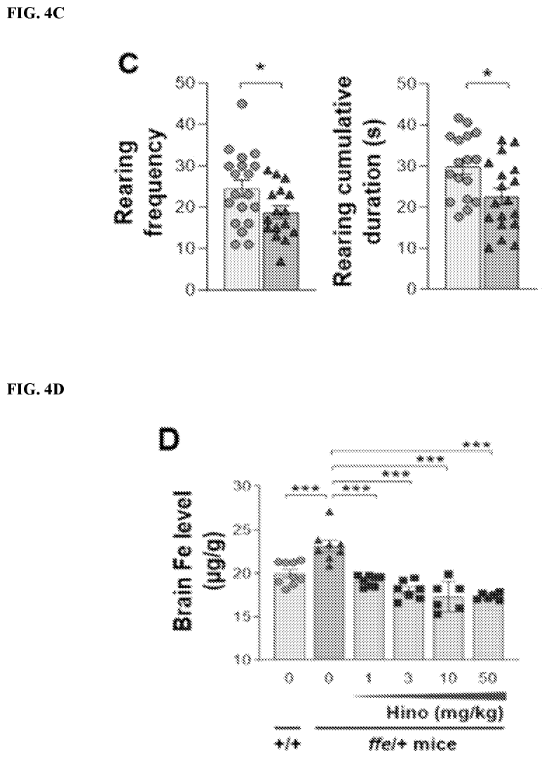

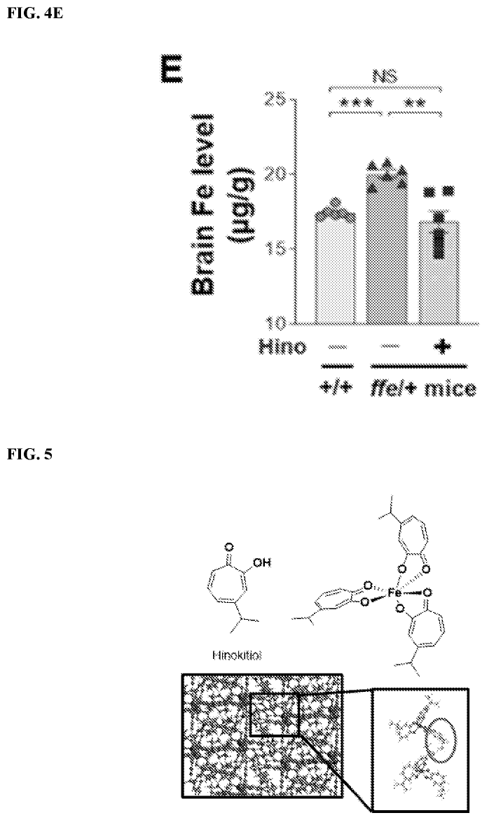

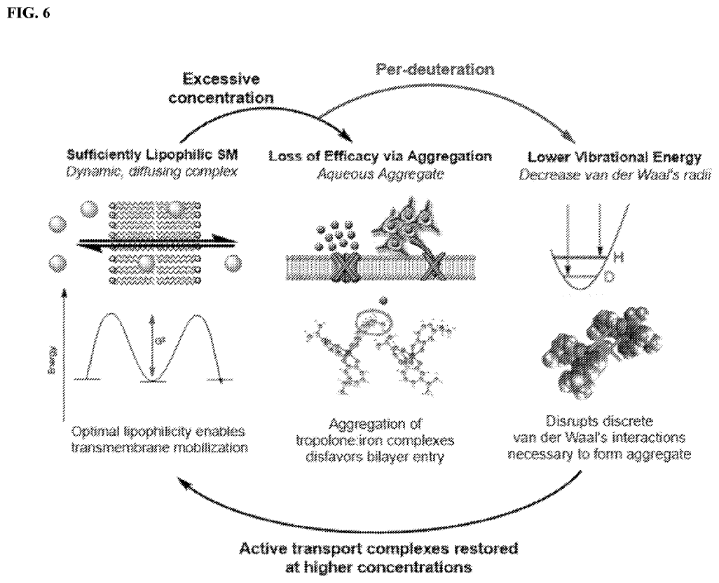

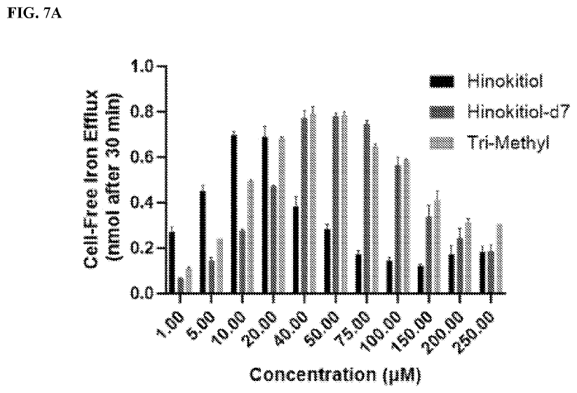

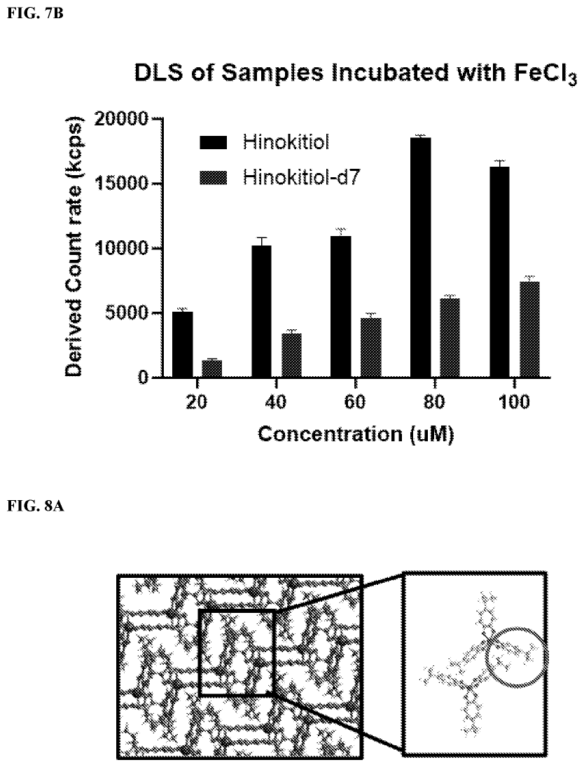

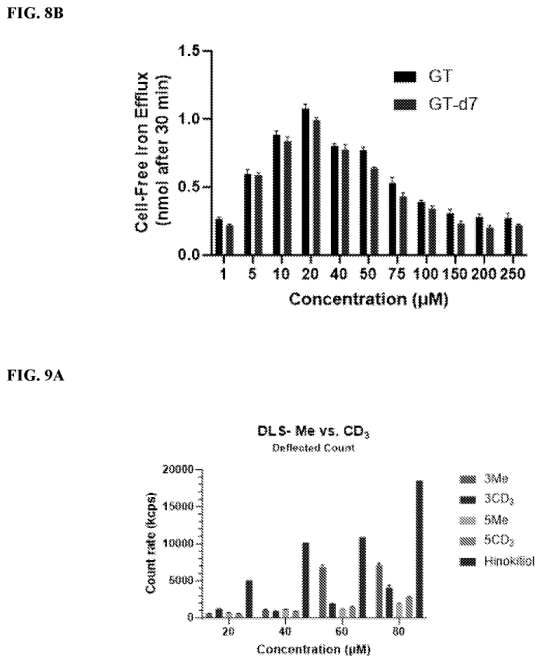

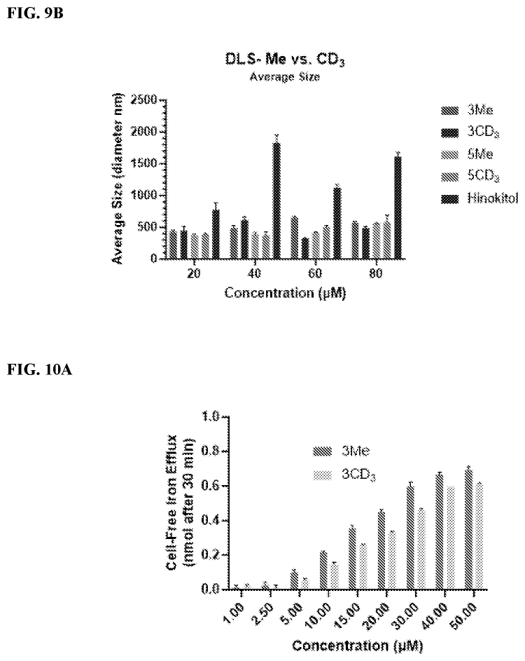

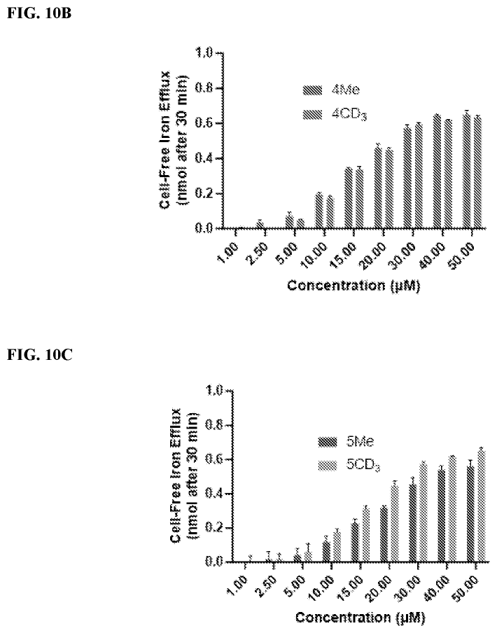

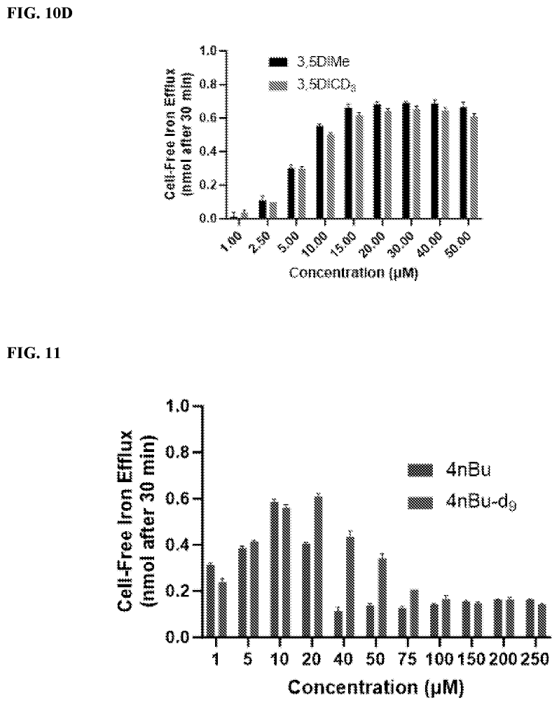

Data for this experiment are shown in FIG. 4A-E

### Animal Care

This study was performed in strict accordance with the recommendations in the Guide for the Care and Use of Laboratory Animals of the National Institutes of Health (Bethesda, MD, USA). The protocol (protocol number: PRO00010742) was approved by the University Committee on Use and Care of Animals (UCUCA) at the University of Michigan. Flatiron (ffe/+) mice were kindly provided by I. E. Zohn (Children's National Medical Center, Washington, DC). All mice used for these studies were on the 129S6/SvEvTac background. Weanling mice were fed a diet (TD120518; Harlan Teklad, Indianapolis, IN, USA) as previously described (Y. A. Seo, J. A. Elkhader, M. Wessling-Resnick, Distribution of manganese and other biometals in flatiron mice. Biometals 29, 147-155 (2016); Y. A. Seo, M. Wessling-Resnick, Ferroportin deficiency impairs manganese metabolism in flatiron mice. FASEB J 29, 2726-2733 (2015)).

### Elevated Plus Maze

This technique is widely used in mice to test anxiety-like behavior and motor activity (A. A. Walf, C. A. Frye, The use of the elevated plus maze as an assay of anxiety-related behavior in rodents. Nat Protoc 2, 322-328 (2007)). The elevated plus maze consists of 2 open arms and 2 closed arms that extend from the central zone platform. Each mouse was placed in the center zone, facing an open arm, and was allowed to explore the maze for 5 min. The time spent and the frequency of entries into the three areas of the apparatus, namely an open arm, a closed arm, and the center zone, as well as total distance traveled on the whole maze, rearing frequency, and duration of movement was recorded and analyzed by EthoVision XT (Noldus).

### Inductively Coupled Plasma-Mass Spectrometry (ICP-MS) Measurement of Brain Fe Level

To characterize the acute and chronic effects of Hino on iron mobilization and distribution, flatiron mice and WT littermates were administered Hino via intraperitoneal (IP) injection. Mice were euthanized at 4 hours or after 1 week, the brain was collected, and the content of iron was determined by ICP-MS (Y. A. Seo, J. A. Elkhader, M. Wessling-Resnick, Distribution of manganese and other biometals in flatiron mice. Biometals 29, 147-155 (2016); Y. A. Seo, M. Wessling-Resnick, Ferroportin deficiency impairs manganese metabolism in flatiron mice. FASEB J 29, 2726-2733 (2015)).

### Example 16—Dynamic Light Scattering Experiments

Dynamic light scattering was performed on a Malvern Zetasizer Nano using disposable cuvettes. Samples were evaluated in a similar buffer to liposome efflux studies (50 mM MES/TRIS, pH to 7.0 using solutions of 1 M MES or 1 M TRIS). Compound at the specified concentration (from 1000× in DMSO), as well as equimolar FeCl₃ (1000× in 0.1 M HCl) were added to this buffer. After addition, these compounds were vortexed for 30 sec, sonicated for one min, then further vortexed for 30 sec before a five-minute equilibration in the Zetasizer at 37 °C. All standard parameters were used, with each graph representing a minimum of thirty 10 second measurements. For count rate analysis, the raw count rate was utilized, accounting for any differences in attenuator position between samples.

### INCORPORATION BY REFERENCE

All US patents and US and PCT patent application publications mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A compound of Formula I: 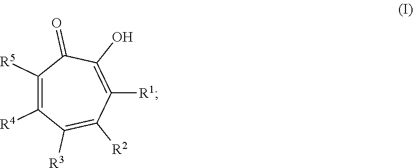
or a pharmaceutically acceptable salt thereof;
wherein:
R¹ is selected from the group consisting of H, C₁-C₅ alkyl, cycloalkyl, heterocycloalkyl, —O—C₁-C₅ alkyl, —O-cycloalkyl, and —O-heterocycloalkyl;
R² is selected from the group consisting of H, C₁-C₅ alkyl, cycloalkyl, heterocycloalkyl, —O—C₁-C₅ alkyl, —O-cycloalkyl, and —O-heterocycloalkyl; and
R³ is selected from the group consisting of H, C₁-C₅ alkyl, cycloalkyl, heterocycloalkyl, —O—C₁-C₅ alkyl, —O-cycloalkyl, and —O-heterocycloalkyl;
R⁴ is selected from the group consisting of H, C₁-C₅ alkyl, cycloalkyl, heterocycloalkyl, —O—C₁-C₅ alkyl, —O-cycloalkyl, and —O-heterocycloalkyl; and
R⁵ is selected from the group consisting of H, halo, C₁-C₅ alkyl, cycloalkyl, heterocycloalkyl, —O—C₁-C₅ alkyl, —O-cycloalkyl, and —O-heterocycloalkyl;
wherein when R¹, R², R³, R₄, or R⁵ is C₁-C₅ alkyl, cycloalkyl, heterocycloalkyl, —O—C₁-C₅ alkyl, —O-cycloalkyl, or —O-heterocycloalkyl, at least one hydrogen atom in said C₁-C₅ alkyl, cycloalkyl, heterocycloalkyl, —O—C₁-C₅ alkyl, —O-cycloalkyl, or —O-heterocycloalkyl is replaced with a deuterium atom; and
wherein at least one of R¹, R², R³, and R⁴ is not H.

2. The compound of claim 1, wherein R¹ is selected from the group consisting of methyl, ethyl, propyl, and butyl.

3. The compound of claim 1, wherein R¹ is selected from the group consisting of CD₃, C₂D₅, C₃D₇, and C₄D₉.

4. The compound of claim 1, wherein R¹ is:

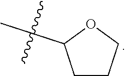 [SMILES: *C1CCCO1]

5. The compound of claim 1, wherein R¹ is:

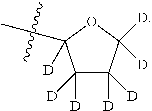

6. The compound of claim 1, wherein R¹ is:

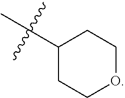 [SMILES: *C1CCOCC1]

7. The compound of claim 1, wherein R¹ is:

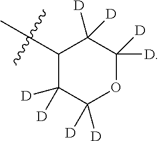

8. The compound of claim 1, wherein R¹ is:

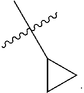 [SMILES: *C1CC1]

9. The compound of claim 1, wherein R¹ is:

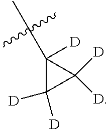

10. The compound of claim 1, wherein R¹ is selected from the group consisting of —OMe, —OEt, —OPr, and —OBu.

11. The compound of claim 1, wherein R¹ is selected from the group consisting of —OCD₃, —OC₂D₅, —OC₃D₇, and —OC₄D₉.

12. The compound of claim 1, wherein R¹ is:

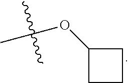 [SMILES: *OC1CCC1]

13. The compound of claim 1, wherein R¹ is:

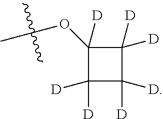

14. The compound of any one of claims 1-13, wherein R² is selected from the group consisting of methyl, ethyl, propyl, and butyl.

15. The compound of any one of claims 1-13, wherein R² is selected from the group consisting of CD₃, C₂D₅, C₃D₇, and C₄D₉.

16. The compound of any one of claims 1-13, wherein R² is:

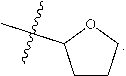 [SMILES: *C1CCCO1]

17. The compound of any one of claims 1-13, wherein R² is:

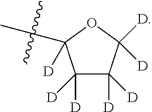

18. The compound of any one of claims 1-13, wherein R² is:

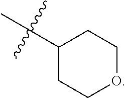 [SMILES: *C1CCOCC1]

19. The compound of any one of claims 1-13, wherein R² is:

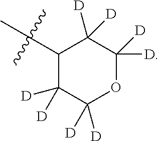

20. The compound of any one of claims 1-13, wherein R² is:

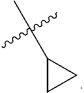 [SMILES: *C1CC1]

21. The compound of any one of claims 1-13, wherein R² is:

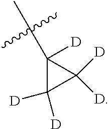

22. The compound of any one of claims 1-13, wherein R² is selected from the group consisting of —OMe, —OEt, —OPr, and —OBu.

23. The compound of any one of claims 1-13, wherein R² is selected from the group consisting of —OCD₃, —OC₂D₅, —OC₃D₇, and —OC₄D₉.

24. The compound of any one of claims 1-13, wherein R² is:

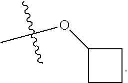 [SMILES: *OC1CCC1]

25. The compound of any one of claims 1-13, wherein R² is:

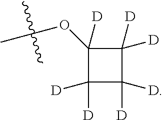

26. The compound of any one of claims 1-25, wherein R³ is selected from the group consisting of methyl, ethyl, propyl, and butyl.

27. The compound of any one of claims 1-25, wherein R³ is selected from the group consisting of CD₃, C₂D₅, C₃D₇, and C₄D₉.

28. The compound of any one of claims 1-25, wherein R³ is:

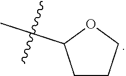 [SMILES: *C1CCCO1]

29. The compound of any one of claims 1-25, wherein R³ is:

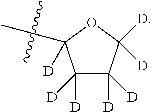

30. The compound of any one of claims 1-25, wherein R³ is:

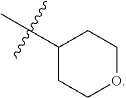 [SMILES: *C1CCOCC1]

31. The compound of any one of claims 1-25, wherein R³ is:

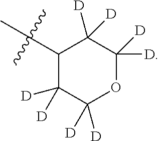

32. The compound of any one of claims 1-25, wherein R³ is:

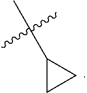 [SMILES: *C1CC1]

33. The compound of any one of claims 1-25, wherein R³ is:

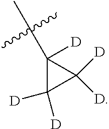

34. The compound of any one of claims 1-25, wherein R³ is selected from the group consisting of —OMe, —OEt, —OPr, and —OBu.

35. The compound of any one of claims 1-25, wherein R³ is selected from the group consisting of —OCD₃, —OC₂D₅, —OC₃D₇, and —OC₄D₉.

36. The compound of any one of claims 1-25, wherein R³ is:

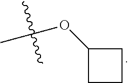 [SMILES: *OC1CCC1]

37. The compound of any one of claims 1-25, wherein R³ is:

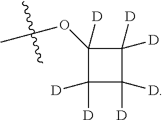

38. The compound of any one of claims 1-37, wherein R⁴ is selected from the group consisting of methyl, ethyl, propyl, and butyl.

39. The compound of any one of claims 1-37, wherein R⁴ is selected from the group consisting of CD₃, C₂D₅, C₃D₇, and C₄D₉.

40. The compound of any one of claims 1-37, wherein R⁴ is:

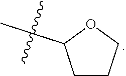 [SMILES: *C1CCCO1]

41. The compound of any one of claims 1-37, wherein R⁴ is:

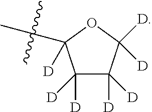

42. The compound of any one of claims 1-37, wherein R⁴ is:

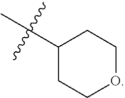 [SMILES: *C1CCOCC1]

43. The compound of any one of claims 1-37, wherein R⁴ is:

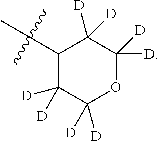

44. The compound of any one of claims 1-37, wherein R⁴ is:

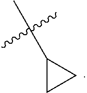 [SMILES: *C1CC1]

45. The compound of any one of claims 1-37, wherein R⁴ is:

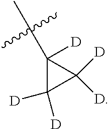

46. The compound of any one of claims 1-37, wherein R⁴ is selected from the group consisting of —OMe, —OEt, —OPr, and —OBu.

47. The compound of any one of claims 1-37, wherein R⁴ is selected from the group consisting of —OCD₃, —OC₂D₅, —OC₃D₇, and —OC₄D₉.

48. The compound of any one of claims 1-37, wherein R⁴ is:

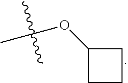 [SMILES: *OC1CCC1]

49. The compound of any one of claims 1-37, wherein R⁴ is:

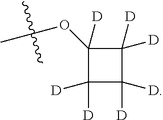

50. The compound of any one of claims 1-49, wherein R⁵ is H.

51. The compound of any one of claims 1-49, wherein R⁵ is halo.

52. The compound of any one of claims 1-49, wherein R⁵ is F.

53. The compound of any one of claims 1-49, wherein R⁵ is selected from the group consisting of methyl, ethyl, propyl, and butyl.

54. The compound of any one of claims 1-49, wherein R⁵ is selected from the group consisting of CD₃, C₂D₅, C₃D₇, and C₄D₉.

55. The compound of any one of claims 1-49, wherein R⁵ is:

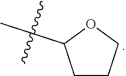 [SMILES: *C1CCCO1]

56. The compound of any one of claims 1-49, wherein R⁵ is:

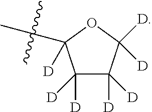

57. The compound of any one of claims 1-49, wherein R⁵ is:

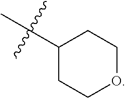 [SMILES: *C1CCOCC1]

58. The compound of any one of claims 1-49, wherein R⁵ is:

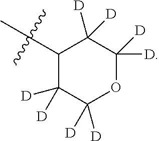

59. The compound of any one of claims 1-49, wherein R⁵ is:

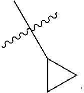 [SMILES: *C1CC1]

60. The compound of any one of claims 1-49, wherein R⁵ is:

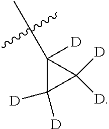

61. The compound of any one of claims 1-49, wherein R⁵ is selected from the group consisting of —OMe, —OEt, —OPr, and —OBu.

62. The compound of any one of claims 1-49, wherein R⁵ is selected from the group consisting of —OCD₃, —OC₂D₅, —OC₃D₇, and —OC₄D₉.

63. The compound of any one of claims 1-49, wherein R⁵ is:

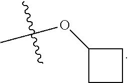 [SMILES: *OC1CCC1]

64. The compound of any one of claims 1-49, wherein R⁵ is:

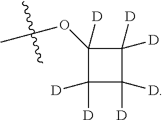

65. The compound of claim 1, wherein the compound is selected from the group consisting of: 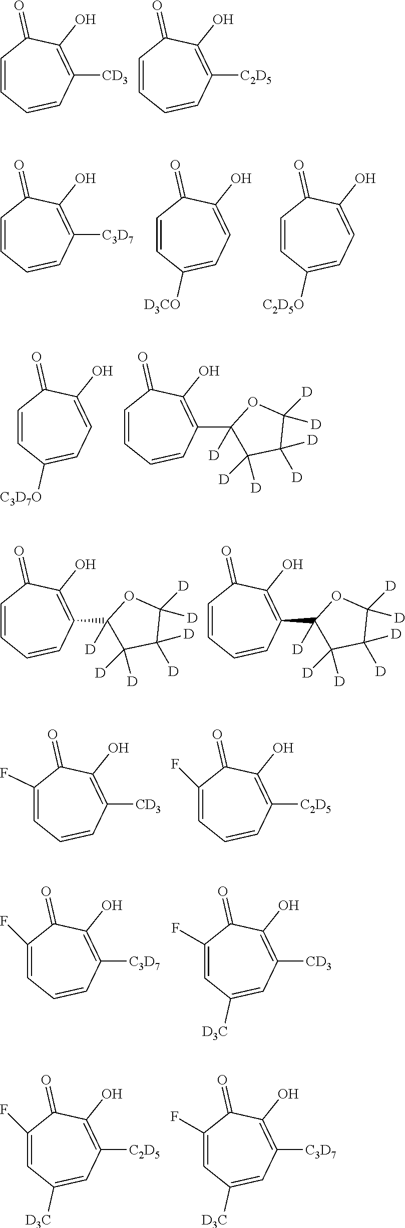 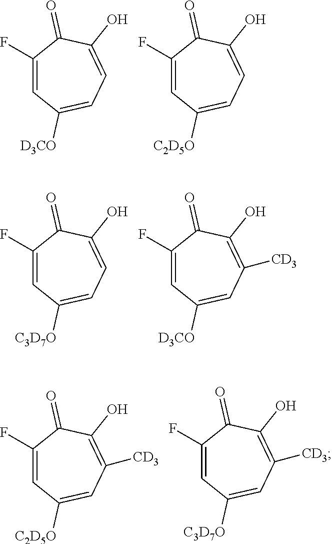
or a pharmaceutically acceptable salt thereof.

66. The compound of claim 1, wherein the compound is selected from the group consisting of: 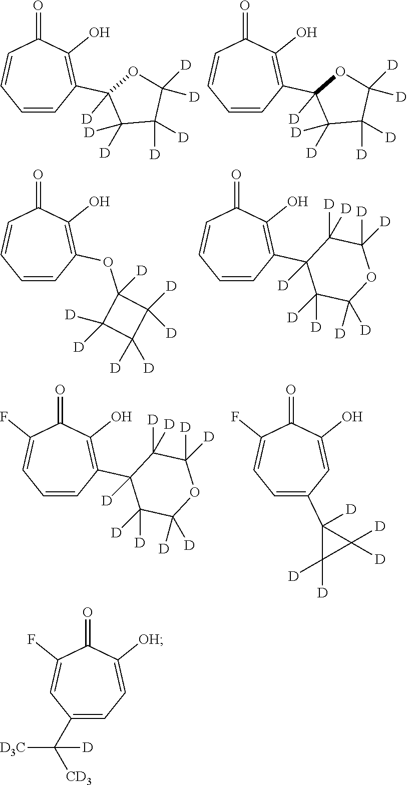
or a pharmaceutically acceptable salt thereof.

67. The compound of claim 1, wherein the compound is selected from the group consisting of: 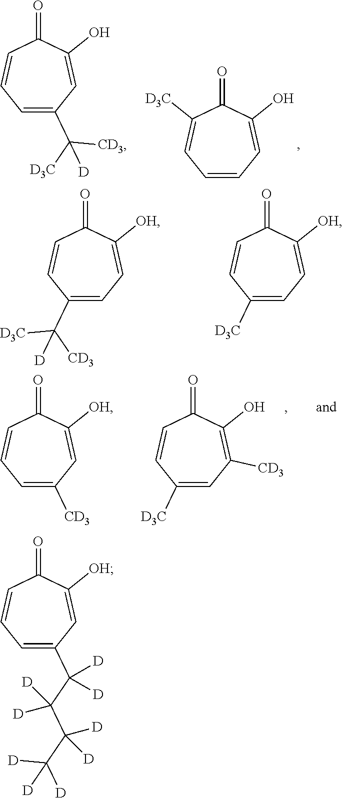
or a pharmaceutically acceptable salt thereof.

68. A pharmaceutical composition, comprising a compound of any one of claims 1-67, or a pharmaceutically acceptable salt thereof; and a pharmaceutically acceptable carrier or excipient.

69. The pharmaceutical composition of claim 68, further comprising an additional therapeutic agent selected from the group consisting of an iron mobilizer and deferiprone.

70. A method of treating a disease or condition characterized by decreased ferroportin, comprising administering to a subject in need thereof a therapeutically effective amount of a compound of any one of claims 1-67, or the pharmaceutical composition of claim 68 or 69.

71. The method of claim 70, wherein the disease or condition characterized by decreased ferroportin is induced ferroportin disease associated with aging.

72. A method of treating a disease or condition characterized by decreased iron transport, comprising administering to a subject in need thereof a therapeutically effective amount of a compound of any one of claims 1-67, or the pharmaceutical composition of claim 68 or 69.

73. A method of treating a disease or condition characterized by a defect or deficiency in an iron transporter, comprising administering to a subject in need thereof a therapeutically effective amount of a compound of any one of claims 1-67, or the pharmaceutical composition of claim 68 or 69.

74. A method of treating a disease or condition characterized by iron accumulation, comprising administering to a subject in need thereof a therapeutically effective amount of a compound of any one of claims 1-67, or the pharmaceutical composition of claim 68 or 69.

75. The method of claim 74, wherein the iron accumulation is iron accumulation in the brain.

76. A method of treating a disease or condition characterized by iron misdistribution, comprising administering to a subject in need thereof a therapeutically effective amount of a compound of any one of claims 1-67, or the pharmaceutical composition of claim 68 or 69.

77. The method of claim 76, wherein the iron misdistribution is iron misdistribution in the brain.

78. The method of any one of claims 72-77, wherein the disease or condition is selected from the group consisting of anemia of inflammation, iron overload, thalassemia, hemochromatosis, atransferrinemia, myelodysplasia, hypochromic microcytic anemia, ferroportin disease, transfusional iron overload, and aceruloplasminemia.

79. A method of treating a neurodegenerative disorder, comprising administering to a subject in need thereof a therapeutically effective amount of a compound of any one of claims 1-67, or the pharmaceutical composition of claim 68 or 69.

80. The method of claim 79, wherein the neurodegenerative disorder is selected from the group consisting of Parkinson's Disease, Alzheimer's Disease, Amyotrophic lateral sclerosis, Friedrich's ataxia, Huntington's Disease, Lewy Body Disease, and Spinal Muscular Atrophy.

81. The method of claim 79, wherein the neurodegenerative disorder is neurodegeneration with brain iron accumulation (NBIA).

82. The method of claim 81, wherein the NBIA is selected from the group consisting of Beta-propeller Protein-associated Neurodegeneration (BPAN), Pantothenate Kinase-associated Neurodegeneration (PKAN), PLA2G6-associated Neurodegeneration (PLAN), Mitochondrial-membrane Protein-associated Neurodegeneration (MPAN), Fatty Acid Hydroxylase-Associated Neurodegeneration (FAHN), COASY Protein-Associated Neurodegeneration (CoPAN), Aceruloplasminemia, Kufor-Rakeb Syndrome (also known as Parkinson's Disease 9 (PARK9)), Neuroferritinopathy, Woodhouse-Sakati Syndrome, and Idiopathic NBIA.

83. The method of any one of claims 70-82, further comprising administering to the subject an effective amount of an additional therapeutic agent selected from the group consisting of an iron mobilizer and deferiprone.
